SEQUENCE LISTING (41 sequences)

SEQ ID NO: 1 - DNA, 33136 bp, synthetic construct
  misc_feature 1..33136: note = HDV with human PRG4
  source 1..33136: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgtcgacccaacgcgttgggagctctccggatccaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatgcatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaattcggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccactcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctgtgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaacgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagaaaaaaaaaaaaaaaaaggccacatgtgctcgagctgcaggtcgcggccctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttggggtaccgaattcctcgagtctagaggagcatgcgacgtcggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 2 - DNA, 31815 bp, synthetic construct
  misc_feature 1..31815: note = HDAd with murine PRG4
  source 1..31815: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgtcgacccaacgcgttgggagctctccggatccaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatgcatggggtggaaaatacttcccgtctgcttgtcgctgctgcttcctgttgtcttgattcaacaggtttcatctcaagagctctcctgcaaaggacgctgcttcgagtccttcgctcgagggagggagtgtgactgtgactcccagtgtaaacagtatggcaagtgctgtgcagattatgacagcttctgtgaagaagtaaaagataacaagaaaaacacccctaaaaagaaacccaatccagaaccgccggctgtggatgaagctggaagcgggctggataatggtgagttcaagctcaccccacctcctcctgaccctcctactactccacacagcaaagtcgccacatctcccaagactacagcagcaaaaccagtaactcctaaacccagtcttgcacctaattctgagacatccaaagaggcatctttagcatccaataaagagacaacagttgaaacgaaagagactactgcaacaaataaacaatcttctgccagtaaaaaaaagacgacttcagttaaagagacacggagtgcagagaaaacatctgataaagatgttgagcccacatctacaactcccaagaactcagctcccactactaccaagaagcctgttactacaaccaaggaatccaagttcctaccactcccacaggagcctgaacccacaactgccaaggaaccacctccaaccaccaagaagcctgaacccaccacccgcaaggagcctgaacccacaactcccaaggagcctgaacccaccacccccaaggagcctgaacccaccactcccaaggagcctgaacccacaactcccaaggaaccacctccaaccaccaagaagcctgaacccaccacccccaaggagcctggacccacaactcccaaggagcctgaacccaccaccaccaaggagcctgaacccaccaccaccaaggagcctgaatccaccactcgcaaggagcctgaacccaccactcccaaggagcctgaacccacaactcccaaggagcctgaacctacaaccctcaaggagcctgaacccacaactcccaaggagcctgaacccaccactcccaaggagcctgaacccacaactcccaaggagcctgaacccacaactcccaaggagcctgaacccaccacgcccaaggagcctgaacccacaactcccaaggagcctgaacccaccacgcccaaggagcctgaacccacaacccccaaggagcctgaacccacaactcccaagaaacctgaacccacaactcccaaggagcctgtacctacaacccccaaggagcctgaacccaccacccccaaggagcctgaacccaccacgcccaaggagcctgaacccacaactcgcaaggagcctgaacctacaactcccaaggagcctgaacctacaacccccaaggagcctgaacccacaactcccaagaagcctgaacccaccaccacatcacccaaaaccactactctgaaagcaacaactcttgcacccaaagtaactgctccagcagaagagattcagaacaaacctgaagaaacaactcctgcatcagaagattctgatgattctaaaacaactctaaaaccacagaagccaaccaaagcacccaagcctaccaaaaagccaaccaaagcacccaagaagcccacctctaccaaaaagccaaagacaccaaaaacaagaaaaccaaaaactacaccagctcctctaaagacgacttcagcaacacctgaactgaataccacccctctagaagtcatgctgccaaccaccaccatccctaaacaaactccaaaccctgaaacagctgaagtaaatccagatcatgaagatgcagatggaggtgaaggagaaaaacctctgattcccgggccccctgtgctattccccacagctattccaggcactgatcttttggccgggagactcaatcgaggcattaacatcaatcccatgccttcagatgagaccaatttatgcaatggtaagccagtggatggactgactacgctgcgcaatgggacattagttgcatttcgaggtcattatttctggatgctgaatccattcagaccaccatctccaccacgcagaattaccgaagtttggggtattccctctcctattgacacagtttttactagatgtaactgtgaaggaaaaactttcttctttaaggattctcagtactggcgcttcaccaatgatgtggtagatcctgggtatcctaaacaaattgtcaaaggctttggaggactaacagggaagatagtggctgctctttcaatagccaagtacaaggacaggcctgaatctgtgtacttcttcaagagaggtggcaacattcagcagtacacttataaacaggagccaatgaagaagtgcacagggaggcggcctgccatcaactactcggtgtacggggaggcagcacaggtcaggaggcgccgctttgagcgtgctgttggacctttccagacacacaccttcaggatccattactcggtgcccatgagagtttcttatcaagacaaaggtttcctccacaatgaagtcaaagtgagtacaatgtggagagggtttccaaatgttgttacttcagcgataacactgcctaacattaggaaacctgatggctatgattactacgccttttccaaagatcaatactataacattgatgtgcctacaagaacagcaagagcaattaccacacgttcagggcagaccctctccaaaatctggtacaactgtccttgaactgattgacaaagaagagtcatcaaaatgaaatgaagaaaacagtgatactttttgacattgaaatacattttattaataaagaatgttgaaatgagcatacctatttaaatacaaaaaaaaaaaaaaaaaaggccacatgtgctcgagctgcaggtcgcggccctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttggggtaccgaattcctcgagtctagaggagcatgcgacgtcggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 3 - DNA, 4215 bp, Homo sapiens
  source 1..4215: mol_type = other DNA
atggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaattcggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccactcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctgtgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaacgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttag

SEQ ID NO: 4 - DNA, 2894 bp, Mus musculus
  source 1..2894: mol_type = other DNA
atggggtggaaaatacttcccgtctgcttgtcgctgctgcttcctgttgtcttgattcaacaggtttcatctcaagagctctcctgcaaaggacgctgcttcgagtccttcgctcgagggagggagtgtgactgtgactcccagtgtaaacagtatggcaagtgctgtgcagattatgacagcttctgtgaagaagtaaaagataacaagaaaaacacccctaaaaagaaacccaatccagaaccgccggctgtggatgaagctggaagcgggctggataatggtgagttcaagctcaccccacctcctcctgaccctcctactactccacacagcaaagtcgccacatctcccaagactacagcagcaaaaccagtaactcctaaacccagtcttgcacctaattctgagacatccaaagaggcatctttagcatccaataaagagacaacagttgaaacgaaagagactactgcaacaaataaacaatcttctgccagtaaaaaaaagacgacttcagttaaagagacacggagtgcagagaaaacatctgataaagatgttgagcccacatctacaactcccaagaactcagctcccactactaccaagaagcctgttactacaaccaaggaatccaagttcctaccactcccacaggagcctgaacccacaactgccaaggaaccacctccaaccaccaagaagcctgaacccaccacccgcaaggagcctgaacccacaactcccaaggagcctgaacccaccacccccaaggagcctgaacccaccactcccaaggagcctgaacccacaactcccaaggaaccacctccaaccaccaagaagcctgaacccaccacccccaaggagcctggacccacaactcccaaggagcctgaacccaccaccaccaaggagcctgaacccaccaccaccaaggagcctgaatccaccactcgcaaggagcctgaacccaccactcccaaggagcctgaacccacaactcccaaggagcctgaacctacaaccctcaaggagcctgaacccacaactcccaaggagcctgaacccaccactcccaaggagcctgaacccacaactcccaaggagcctgaacccacaactcccaaggagcctgaacccaccacgcccaaggagcctgaacccacaactcccaaggagcctgaacccaccacgcccaaggagcctgaacccacaacccccaaggagcctgaacccacaactcccaagaaacctgaacccacaactcccaaggagcctgtacctacaacccccaaggagcctgaacccaccacccccaaggagcctgaacccaccacgcccaaggagcctgaacccacaactcgcaaggagcctgaacctacaactcccaaggagcctgaacctacaacccccaaggagcctgaacccacaactcccaagaagcctgaacccaccaccacatcacccaaaaccactactctgaaagcaacaactcttgcacccaaagtaactgctccagcagaagagattcagaacaaacctgaagaaacaactcctgcatcagaagattctgatgattctaaaacaactctaaaaccacagaagccaaccaaagcacccaagcctaccaaaaagccaaccaaagcacccaagaagcccacctctaccaaaaagccaaagacaccaaaaacaagaaaaccaaaaactacaccagctcctctaaagacgacttcagcaacacctgaactgaataccacccctctagaagtcatgctgccaaccaccaccatccctaaacaaactccaaaccctgaaacagctgaagtaaatccagatcatgaagatgcagatggaggtgaaggagaaaaacctctgattcccgggccccctgtgctattccccacagctattccaggcactgatcttttggccgggagactcaatcgaggcattaacatcaatcccatgccttcagatgagaccaatttatgcaatggtaagccagtggatggactgactacgctgcgcaatgggacattagttgcatttcgaggtcattatttctggatgctgaatccattcagaccaccatctccaccacgcagaattaccgaagtttggggtattccctctcctattgacacagtttttactagatgtaactgtgaaggaaaaactttcttctttaaggattctcagtactggcgcttcaccaatgatgtggtagatcctgggtatcctaaacaaattgtcaaaggctttggaggactaacagggaagatagtggctgctctttcaatagccaagtacaaggacaggcctgaatctgtgtacttcttcaagagaggtggcaacattcagcagtacacttataaacaggagccaatgaagaagtgcacagggaggcggcctgccatcaactactcggtgtacggggaggcagcacaggtcaggaggcgccgctttgagcgtgctgttggacctttccagacacacaccttcaggatccattactcggtgcccatgagagtttcttatcaagacaaaggtttcctccacaatgaagtcaaagtgagtacaatgtggagagggtttccaaatgttgttacttcagcgataacactgcctaacattaggaaacctgatggctatgattactacgccttttccaaagatcaatactataacattgatgtgcctacaagaacagcaagagcaattaccacacgttcagggcagaccctctccaaaatctggtacaactgtccttgaactgattgacaaagaagagtcatcaaaatgaaatgaagaaaacagtgatactttttgacattgaaatacattttattaataaagaatgttgaaatgagcatacctatttaaataca

SEQ ID NO: 5 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTCDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 6 - protein, 925 aa, Mus musculus
MGWKILPVCLSLLLPVVLIQQVSSQELSCKGRCFESFARGRECDCDSQCKQYGKCCADYDSFCEEVKDNKKNTPKKKPNPEPPAVDEAGSGLDNGEFKLTPPPPDPPTTPHSKVATSPKTTAAKPVTPKPSLAPNSETSKEASLASNKETTVETKETTATNKQSSASKKKTTSVKETRSAEKTSDKDVEPTSTTPKNSAPTTTKKPVTTTKESKFLPLPQEPEPTTAKEPPPTTKKPEPTTRKEPEPTTPKEPEPTTPKEPEPTTPKEPEPTTPKEPPPTTKKPEPTTPKEPGPTTPKEPEPTTTKEPEPTTTKEPESTTRKEPEPTTPKEPEPTTPKEPEPTTLKEPEPTTPKEPEPTTPKEPEPTTPKEPEPTTPKEPEPTTPKEPEPTTPKEPEPTTPKEPEPTTPKEPEPTTPKKPEPTTPKEPVPTTPKEPEPTTPKEPEPTTPKEPEPTTRKEPEPTTPKEPEPTTPKEPEPTTPKKPEPTTTSPKTTTLKATTLAPKVTAPAEEIQNKPEETTPASEDSDDSKTTLKPQKPTKAPKPTKKPTKAPKKPTSTKKPKTPKTRKPKTTPAPLKTTSATPELNTTPLEVMLPTTTIPKQTPNPETAEVNPDHEDADGGEGEKPLIPGPPVLFPTAIPGTDLLAGRLNRGININPMPSDETNLCNGKPVDGLTTLRNGTLVAFRGHYFWMLNPFRPPSPPRRITEVWGIPSPIDTVFTRCNCEGKTFFFKDSQYWRFTNDVVDPGYPKQIVKGFGGLTGKIVAALSIAKYKDRPESVYFFKRGGNIQQYTYKQEPMKKCTGRRPAINYSVYGEAAQVRRRRFERAVGPFQTHTFRIHYSVPMRVSYQDKGFLHNEVKVSTMWRGFPNVVTSAITLPNIRKPDGYDYYAFSKDQYYNIDVPTRTARAITTRSGQTLSKIWYNCP

SEQ ID NO: 7 - DNA, 29265 bp, synthetic construct
  misc_feature 1..29265: note = helper-dependent adenoviral vector containing human II-1Ra
  source 1..29265: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgtcgacccaacgcgttgggagctctccggatccaagcttgttgggtcgaagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaaggatcccgcggccgcctactcgtcctcctggaagtagaatttggtgaccatgacgccttcgtcaggcatattggtgaggctgacgggctggtcagcttccatcgctgtgcagaggaaccaaccggggcaggcggcagactcaaaactggtggtggggccactgtctgagcggatgaaggcgaagcgcttgtcctgctttctgttctcgctcaggtcagtgatgttaactgcctccagctggagtctggtctcatcaccagacttgacacaggacaggcacatcttccctccatggattcccaagaacagagcatgaggctcaatgggtaccacatctatcttttcttctaaattgacatttggtccttgcaagtatccagcaactagttggttgttcctcagatagaaggtcttctggttaacatcccagattctgaaggcttgcatcttgctggattttctcccagagggtcggcagatcgtctctgaatggaacaggaagaggaggagagtgattaggtgactgcggaggcctctgcagatttccataccggtggatccatggctctgtctcaggtcagtataggagctttgatgtgaagtcagccaagaacagctgaacactacttcctctgaggcccttttataggagggattgcttcctgtgaataataggaggatattgtccacatccagtaaagaggaaatccccaactgcatccaaaaagttttctgggaatatccactgctgcaggagtggaaagtccccagtggaaagtccccagtggaaagtccccagtggaaagtccccagtggaaagtccccagaatttcgacgtctagaggagcatgcgacgtcggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 8 - DNA, 29612 bp, synthetic construct
  misc_feature 1..29612: note = helper-dependent adenoviral vector containing murine II-1Ra
  source 1..29612: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgacgtcgcatgctcctctagactcgaggaattcggtaccccgggttcgaaatcgataagcttggatcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaggatctgcgatcgctgaattctggggactttccactggggactttccactggggactttccactggggactttccactggggactttccactcctgcagcagtggatattcccagaaaactttttggatgcagttggggatttcctctttactggatgtggacaatatcctcctattattcacaggaagcaatccctcctataaaagggcctcagaggaagtagtgttcagctgttcttggctgacttcacatcaaagctcctatactgacctgagacagagccatgaattccgtttttttttttttttttggaaatatgagggtttttccgcttctgacagtggaacggaatgacagcagcacaggctggtgaatgactactttctttataagcaaccaccttgagcctgaaatggcagtcgctagtctctattgccttgctgtggcctcgggatggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatagtctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttagatccggagagctcccaacgcgttgggtcgacggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 9 - DNA, 29273 bp, synthetic construct
  misc_feature 1..29273: note = helper-dependent adenoviral vector containing equine II-1Ra
  source 1..29273: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgacgtcgcatgctcctctagactcgaggaattcggtaccccgggttcgaaatcgataagcttggatccggagagctcgataacatttaaatggatctaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcaatagcatcacaaatttcacaaataaagcatttttttcactgcattctagttgtggtttgtccaaactcatcaatgtatcttatcatgtcgagctaggatcccgcggccgcctactggtcctcctggaggtagaacttggtgaccatgaaggactctttgggcttgttggtgaggctgacgggccggtctgcctcctgcgccgtgcagaggaaccagccagggcaggcggcagactcgaagctggtggtggggccactgtttgagcggatgaaggtgaagcgcttgttctcctccttgttcttgctcaggtcagtgatgttaactgcctccaattggaacctaatctcatcaccagacttgacacaggccaggcacagcttcctcccatggagtcccaggaatagagcatcaggctcaatgggcaccacatctatcttctcttgtaatttagtatttgattcttgcaagtatccagcaactagttggttattcctcatgtagaaggtcttctggttaacatcccagattctgaaggcttgcatcttgcagggtctcttccccaaagggtggcaggctgtctctgagtagagcaagaaaaggaggagagagattaggtgtctgacagaacgcctgcggatttccataccggtggatccatggctctgtctcaggtcagtataggagctttgatgtgaagtcagccaagaacagctgaacactacttcctctgaggcccttttataggagggattgcttcctgtgaataataggaggatattgtccacatccagtaaagaggaaatccccaactgcatccaaaaagttttctgggaatatccactgctgcaggagtggaaagtccccagtggaaagtccccagtggaaagtccccagtggaaagtccccagtggaaagtccccagaatttcgacggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 10 - DNA, 534 bp, Homo sapiens
  source 1..534: mol_type = other DNA
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 11 - DNA, 535 bp, Mus musculus
  source 1..535: mol_type = other DNA
atggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaat

SEQ ID NO: 12 - DNA, 534 bp, unidentified
  misc_feature 1..534: note = Equus sp.
  source 1..534: mol_type = other DNA
atggaaatccgcaggcgttctgtcagacacctaatctctctcctccttttcttgctctactcagagacagcctgccaccctttggggaagagaccctgcaagatgcaagccttcagaatctgggatgttaaccagaagaccttctacatgaggaataaccaactagttgctggatacttgcaagaatcaaatactaaattacaagagaagatagatgtggtgcccattgagcctgatgctctattcctgggactccatgggaggaagctgtgcctggcctgtgtcaagtctggtgatgagattaggttccaattggaggcagttaacatcactgacctgagcaagaacaaggaggagaacaagcgcttcaccttcatccgctcaaacagtggccccaccaccagcttcgagtctgccgcctgccctggctggttcctctgcacggcgcaggaggcagaccggcccgtcagcctcaccaacaagcccaaagagtccttcatggtcaccaagttctacctccaggaggaccagtag

SEQ ID NO: 13 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 14 - protein, 178 aa, Mus musculus
MEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 15 - protein, 177 aa, unidentified
  REGION 1..177: note = Equus sp.
MEIRRRSVRHLISLLLFLLYSETACHPLGKRPCKMQAFRIWDVNQKTFYMRNNQLVAGYLQESNTKLQEKIDVVPIEPDALFLGLHGRKLCLACVKSGDEIRFQLEAVNITDLSKNKEENKRFTFIRSNSGPTTSFESAACPGWFLCTAQEADRPVSLTNKPKESFMVTKFYLQEDQ

SEQ ID NO: 16 - DNA, 24 bp, synthetic construct
  misc_feature 1..24: note = PRG4-F Primer
  source 1..24: mol_type = other DNA
acttcagctaaagagacacggagt

SEQ ID NO: 17 - DNA, 27 bp, synthetic construct
  misc_feature 1..27: note = PRG4-R Primer
  source 1..27: mol_type = other DNA
gttcaggtggttccttggttgtagtaa

SEQ ID NO: 18 - DNA, 20 bp, synthetic construct
  misc_feature 1..20: note = Sox9-F Primer
  source 1..20: mol_type = other DNA
aagccacacgtcaagcgacc

SEQ ID NO: 19 - DNA, 20 bp, synthetic construct
  misc_feature 1..20: note = Sox9-R Primer
  source 1..20: mol_type = other DNA
gtgctgctgatgccgtaact

SEQ ID NO: 20 - DNA, 26 bp, synthetic construct
  misc_feature 1..26: note = Col2a1-F Primer
  source 1..26: mol_type = other DNA
gctcatccagggctccaatgatgtag

SEQ ID NO: 21 - DNA, 23 bp, synthetic construct
  misc_feature 1..23: note = Col2a1-R Primer
  source 1..23: mol_type = other DNA
cgggaggtcttctgtgatcggta

SEQ ID NO: 22 - DNA, 21 bp, synthetic construct
  misc_feature 1..21: note = Gapdh-F Primer
  source 1..21: mol_type = other DNA
gcaagagaggccctatcccaa

SEQ ID NO: 23 - DNA, 23 bp, synthetic construct
  misc_feature 1..23: note = Gapdh-R Primer
  source 1..23: mol_type = other DNA
ctccctaggcccctcctgttatt

SEQ ID NO: 24 - DNA, 24 bp, synthetic construct
  misc_feature 1..24: note = Vegf-F Primer
  source 1..24: mol_type = other DNA
tggacttgtgttgggaggaggatg

SEQ ID NO: 25 - DNA, 23 bp, synthetic construct
  misc_feature 1..23: note = Vegf-R Primer
  source 1..23: mol_type = other DNA
gcctcttcttccaccaccgtgtc

SEQ ID NO: 26 - DNA, 24 bp, synthetic construct
  misc_feature 1..24: note = Mmp13-F Primer
  source 1..24: mol_type = other DNA
gcaatctttctttggcttagaggt

SEQ ID NO: 27 - DNA, 22 bp, synthetic construct
  misc_feature 1..22: note = Mmp13-R Primer
  source 1..22: mol_type = other DNA
ggtgttttgggatgcttagggt

SEQ ID NO: 28 - DNA, 20 bp, synthetic construct
  misc_feature 1..20: note = Col10a1-F Primer
  source 1..20: mol_type = other DNA
aaagcttacccagcagtagg

SEQ ID NO: 29 - DNA, 20 bp, synthetic construct
  misc_feature 1..20: note = Col10a1-R Primer
  source 1..20: mol_type = other DNA
acgtactcagaggagtagag

SEQ ID NO: 30 - DNA, 24 bp, synthetic construct
  misc_feature 1..24: note = GAPDH_F Primer
  source 1..24: mol_type = other DNA
ataccaggaaatgagcttgacaaa

SEQ ID NO: 31 - DNA, 20 bp, synthetic construct
  misc_feature 1..20: note = GAPDH_R Primer
  source 1..20: mol_type = other DNA
tgaaggtcggagtcaacgga

SEQ ID NO: 32 - DNA, 26 bp, synthetic construct
  misc_feature 1..26: note = VEGF_F Primer
  source 1..26: mol_type = other DNA
gatcggtgacagtcactagcttatct

SEQ ID NO: 33 - DNA, 22 bp, synthetic construct
  misc_feature 1..22: note = VEGF_R Primer
  source 1..22: mol_type = other DNA
tacacacaaatacaagttgcca

SEQ ID NO: 34 - DNA, 29 bp, synthetic construct
  misc_feature 1..29: note = MMP13_F Primer
  source 1..29: mol_type = other DNA
tgcccttcttcacacagacactaacgaaa

SEQ ID NO: 35 - DNA, 29 bp, synthetic construct
  misc_feature 1..29: note = MMP13_R Primer
  source 1..29: mol_type = other DNA
ggccacatctactattcttaccactgctc

SEQ ID NO: 36 - DNA, 22 bp, synthetic construct
  misc_feature 1..22: note = COL10A1_F Primer
  source 1..22: mol_type = other DNA
gcccactacccaagaccaagac

SEQ ID NO: 37 - DNA, 21 bp, synthetic construct
  misc_feature 1..21: note = COL10A1_R Primer
  source 1..21: mol_type = other DNA
gacccctctcacctggacgac

SEQ ID NO: 38 - DNA, 20 bp, synthetic construct
  misc_feature 1..20: note = HIF3A_F Primer
  source 1..20: mol_type = other DNA
ggctgttccgcctacgagta

SEQ ID NO: 39 - DNA, 19 bp, synthetic construct
  misc_feature 1..19: note = HIF3A_R Primer
  source 1..19: mol_type = other DNA
agcaaggtggatgctcttg

SEQ ID NO: 40 - DNA, 24 bp, synthetic construct
  misc_feature 1..24: note = Fwd Primer
  source 1..24: mol_type = other DNA
tctctttatgaggagttgtggccc

SEQ ID NO: 41 - DNA, 24 bp, synthetic construct
  misc_feature 1..24: note = Reverse primer
  source 1..24: mol_type = other DNA
cgacaacaccacggaattgtcagt

(19) United States
(12) Patent Application Publication
(10) Pub. No.: US 2024/0102049 A1
(43) Pub. Date: Mar. 28, 2024

(54) Helper-dependent adenoviral gene therapy delivery and expression system

(71) Applicant: Baylor College of Medicine, Houston, TX (US)

(72) Inventors: Merry RUAN, South San Francisco, CA (US); Kilian GUSE, Berlin (DE); Brendan LEE, Houston, TX (US)

(21) Appl. No.: 18/352,471

(22) Filed: Jul. 14, 2023

Related U.S. Application Data
(63) Continuation of application No. 17/711,647, filed on Apr. 1, 2022, now Pat. No. 11,746,359.
Application No. 17/711,647 is a continuation of application No. 17/358,904, filed on Jun. 25, 2021.
Application No. 17/358,904 is a continuation of application No. 14/763,326, filed on Jul. 24, 2015, now abandoned.
(60) Provisional application No. 61/756,516, filed on Jan. 25, 2013.

(51) Int. Cl. C12N 15/86; A61K 48/00; A61P 19/02; C07K 14/47; C07K 16/28

(52) U.S. Cl. CPC C12N 15/86; A61K 48/0058; A61P 19/02; C07K 14/4725; C07K 16/2866

(57) ABSTRACT

The present invention relates to gene therapy delivery and expression systems comprising at least one helper-dependent adenoviral vector containing a nucleic acid sequence encoding for proteoglycan 4 (PRG4) or a biologically active fragment thereof. The invention further relates to a pharmaceutical composition comprising a therapeutically effective amount of at least one helper-dependent adenoviral vector containing said nucleic acid sequence encoding for proteoglycan 4 (PRG4), or a homolog thereof from any other species, or a biologically active fragment thereof. The invention also relates to the use of the novel gene therapy delivery and expression system according to the invention for use in the prevention and/or treatment of camptodactyly-arthropathy-coxa vara-pericarditis (CACP), or a musculoskeletal disorder such as a joint disorder or Joint disease.

### CROSS REFERENCE TO RELATED APPLICATIONS

In accordance with 37 C.F.R. § 176, a claim of priority is included in an Application Data Sheet filed concurrently herewith. The present application is a continuation application of U.S. patent application Ser. No. 17/711,647, filed Apr. 1, 2022, which is a continuation application of U.S. patent application Ser. No. 17/358,904, filed Jun. 25, 2021, which is a continuation application of U.S. patent application Ser. No. 14/763,326, filed Jul. 24, 2015, which is a national stage filing in accordance with 35 U.S.C. § 371 of International Patent Application No. PCT/IB2014/000071, filed Jan. 27, 2014, which claims the benefit of U.S. Provisional Patent Application No. 61/756,516, filed Jan. 25, 2013, the entire contents of each of which are incorporated herein by reference.

### REFERENCE TO SEQUENCE LISTING SUBMITTED ELECTRONICALLY

The contents of the electronic sequence listing “GEQB-001_04US_SeqList_ST26.xml”, which was created on Jul. 6, 2023, and is 214,208 bytes, are hereby incorporated by reference in its entirety.

### FIELD OF THE INVENTION

The present invention relates to gene therapy delivery and expression systems comprising at least one helper-dependent adenoviral vector containing a nucleic acid sequence encoding for proteoglycan 4 (PRG4) or a biologically active fragment thereof. The invention further relates to a pharmaceutical composition comprising a therapeutically effective amount of at least one helper-dependent adenoviral vector containing said nucleic acid sequence encoding for proteoglycan 4 (PRG4), or a homolog thereof from any other species, or a biologically active fragment thereof. The invention also relates to the use of the novel gene therapy delivery and expression system according to the invention for use in the prevention and/or treatment of camptodactyly-arthropathy-coxa vara-pericarditis (CACP), or a musculoskeletal disorder such as a joint disorder or joint disease.

### BACKGROUND OF THE INVENTION

Musculoskeletal conditions are the most common chronic conditions, affecting nearly one third of the human population. Musculoskeletal conditions are defined as conditions of the bones, muscles and their attachments such as joints, tendons and ligaments. They consist of a variety of different diseases that cause pain or discomfort in the bones, joints, tendons, ligaments, muscles or surrounding structures. Musculoskeletal disorders range from back pain to rheumatoid arthritis, and gout, and include different types of arthritis, tendinitis and musculoskeletal pain. Furthermore, musculoskeletal diseases or disorders include, but are not limited to arthropathies, all types of arthritis, including arthritis-related disorders, osteoarthritis, rheumatoid arthritis, gout and pseudo-gout, septic arthritis, psoriatic arthritis, ankylosing spondylitis, juvenile idiopathic arthritis. Still's disease, Reiter's syndrome, or tendinopathies including tendonitis, tendinosis, tenosynovitis; synovial disorders including synovitis; Bursa disorders including bursitis; equine musculoskeletal disorders including bone spavin, navicular syndrome, osselet.

In addition, there are heritable disorders such as CAPC (camptodactyly-arthropathy-coxa vara-pericarditis) syndrome that have their origin in a non-functional PRG4 gene. The disorder results in synoviocyte hyperplasia and early onset osteoarthritis, the principal pathological features of the CAPC syndrome.

Osteoarthritis (OA) is an age-related or post-traumatic degenerative disease of the joint that is characterized by loss of articular cartilage, chondrocyte proliferation and hypertrophic differentiation, subchondral bone remodelling, inflammation, and finally, osteophyte formation (K. Johnson et al., A Stem Cell-Based Approach to Cartilage Repair. Science (New York, NY) 336, 717 (Jun. 10, 2012)). It is among the leading causes of chronic disability (Matthews, G. L., and Hunter, D. J. (2011), Emerging drugs for osteoarthritis. Expert Opin. Emerging Drugs 1-13.). Surprisingly, given the impact of OA, relatively few genetic mouse models have been developed to provide insights into potential protective mechanisms that can modify the development of osteoarthritis. To date, most have been loss-of-function genetic models of cartilage degrading enzymes such as ADAMTS5 and MMP13 (F. Echtermeyer et al., Syndecan-4 regulates ADAMTS-5 activation and cartilage breakdown in osteoarthritis. Nature Medicine, 1 (Mar. 30, 2102); T. Saito et al., Transcriptional regulation of endochondral ossification by HIF-2a during skeletal growth and osteoarthritis development. Nature Medicine 16, 678 (Jun. 23, 2010); R. M. Borzi et al., Matrix metalloproteinase 13 loss associated with impaired extracellular matrix remodeling disrupts chondrocyte differentiation by concerted effects on multiple regulatory factors. Arthritis &amp; Rheumatism 62, 2370 (May 13, 2010); J. D. Kay et al., Intra-articular gene delivery and expression of interleukin-1Ra mediated by self-complementary adeno-associated virus. The journal of gene medicine 11, 605 (July, 2009)). Mice with loss of function mutation in Hif2a are also protected from osteoarthritis development, highlighting the importance of the hypoxia pathway in cartilage homeostasis. Unfortunately, despite significant investment, the development of inhibitors of such pathways has not proven effective in the clinical setting.

Interestingly, loss-of-function mutations in proteoglycan 4 (PRG4) in humans cause Camptodactyly-Arthropathy-Coxa Vara-Pericarditis Syndrome (J. Marcelino et al., CACP, encoding a secreted proteoglycan, is mutated in camptodactyly-arthropathy-coxa vara-pericarditis syndrome. Nature genetics 23, 319 (November, 1999)), which is characterized by early onset osteoarthritis. In addition, genetic knockout of PRG4 in mice also results in early osteoarthritis development (D. K. Rhee et al., The secreted glycoprotein lubricin protects cartilage surfaces and inhibits synovial cell overgrowth, J Clin Invest 115, 622 (March, 2005); J. M. Coles et al., Loss of cartilage structure, stiffness, and frictional properties in mice lacking PRG4. Arthritis & amp; Rheumatism 62, 1666 (Jul. 1, 2010)).

PRG4 is also known as lubricin or superficial zone protein or megakaryocyte stimulating factor precursor. It is a component of the cartilage extracellular matrix and synovial fluid (D. K. Rhee et al., The secreted glycoprotein lubricin protects cartilage surfaces and inhibits synovial cell overgrowth, J Clin Invest 115, 622 (March, 2005)). PRG4 is present in synovial fluid and on the surface (superficial layer) of articular cartilage and therefore plays an important role in joint lubrication and synovial homeostasis. Unlike previous osteoarthritis targets, it is a secreted protein produced by superficial zone chondrocytes of the articular cartilage and by synovial lining cells in mammals (D, K. Rhee et al., The secreted glycoprotein lubricin protects cartilage surfaces and inhibits synovial cell overgrowth. J Clin Invest 115, 622 (March, 2005)). The PRG4 gene encodes for glycoprotein of approximately 345 kDa. PRG4 provides synovial fluid with the ability to dissipate strain energy under load and its recombinant protein has been reported to exert chondroprotective effects during the progression of OA in rats (G. D. Jay, J. R. Torres, M. L. Warman, M. C. Lederer, K. S. Breuer, The role of lubricin in the mechanical behavior of synovial fluid. Proc Natl Acad Sci USA 104, 6194 (Apr. 10, 2007); C. R. Flannery et al., Prevention of cartilage degeneration in a rat model of osteoarthritis by intraarticular treatment with recombinant lubricin. Arthritis &amp; Rheumatism 60, 840 (April, 2009)). However, the long-term biological effects of PRG4 over-expression and the molecular mechanism of its potential therapeutic benefits are still poorly understood.

U.S. Pat. No. 6,743,774 A describes a gene therapy approach by administering to a mammal a nucleic acid encoding a therapeutic lubricating polypeptide, such as a lubricating fragment of megakaryocyte stimulating factor precursor by standard vectors and/or gene delivery systems. The gene delivery systems described include liposomes, receptor-mediated delivery systems, naked DNA, viral vectors such as herpes viruses, retro viruses, adenoviruses and adeno-associated viruses. U.S. Pat. No. 7,393,029 A describes polynucleotides for use in gene therapy encoding for recombinant lubricin.

Although some approaches suggest gene therapy for treating or preventing joint disorders such as osteoarthritis, no curative treatments are currently available. Medical treatment is mostly aimed at alleviating the symptoms using analgesic drugs rather than establishing worn away cartilage. An analgesic treatment usually involves steroids and non-steroidal anti-inflammatory drugs (NSAIDS), which have shown efficacy in the treatment of osteoarthritis for some decades. However, while these drugs can suppress joint inflammation, many of them are known to have deteriorating effects on the cartilage, which further worsens the underlying process of osteoarthritis development. Hyaluronic acid, which restores viscoelasticity and lubrication of the joints, has also been widely used. Furthermore, polysulphated glycosaminoglycans injected into the joint or intramuscularly as well as orally administered glucosamine and chondroitin sulphate have been used in the treatment for osteoarthritis, however, the efficacy has not been proven in large randomized trials. Thus, currently used therapies have only limited efficacy in the treatment of joint disorders such as osteoarthritis and their success often depends on the severity of the case. Moreover, these drugs must be administered frequently; sometimes in combination with each other. However, frequent drug injections into the joint are laborious, bear the risk for infections, cause stress for the patient and are costly. It follows that there is a clear and yet unmet medical need for more efficacious and sustained treatments that are at the same time also cost effective in the long run.

The role of PRG4 in joint disorders has been discussed. In addition, during osteoarthritis, interleukin-1 (Il-1) functions as a central mediator of inflammation (Daheshia, M., and YAO, J. Q. (2008). The Interleukin 1β Pathway in the Pathogenesis of Osteoarthritis, J Rheumatol 35, 2306.). Moreover, Il-1 strongly inhibits cartilage matrix synthesis and can trigger matrix breakdown (Evans, C. H., Gouze, J. N. Gouze, E., Robbins, P. D, and Ghivizzani, S. C. (2004)). Osteoarthritis gene therapy, Gene Ther 11, 379-389). To neutralize the effect of Il-1 on synovial inflammation, treatment with interleukin-1 receptor antagonist (Il-1Ra) constitutes a promising concept in the therapy of osteoarthritis (Evans, C. H., Gauze, J. N., Gauze, E., Robbins, P. D., and Ghivizzani. S. C. (2004). Osteoarthritis gene therapy. Gene Ther 11, 379-389; Caron J P et al. Chondroprotective effect of intraarticular injections of interleukin-1 receptor antagonist in experimental osteoarthritis. Suppression of collagenase-1 expression. Arthritis Rheum 1996; 39: 1535-1544)). On nucleic acid level, Il-1Ra is considerably conserved among mammalian species. For example, the cDNA sequences of human Il-1Ra (Accession no: NM_173842) shares 82% homology with the murine variant (Accession no: NM_031167), 84% with the equine variant (Accession no: NM_001082525), 84% with the canine variant (Accession no: NM_001003096), 84% with the lapine variant (Accession no: NM_001082770) and 82% with the bovine variant (Accession no: NM_174357).

Although gene therapy approaches using various gene therapy vectors are known, there is a need for a gene therapy delivery and expression system, which allows for the specific delivery of a therapeutic amount of an active agent to its target. In addition, the active agent shall exhibit its therapeutic effects for a prolonged amount of time. Adeno-associated viruses (AAV) are among the most widely used gene therapy vectors and have shown efficient transduction and long-term transgene expression in many tissues. AAVs have also been used in gene therapy approaches for joints. However, AAV transduction efficiency in joints has never been directly compared to transduction efficiency of other viral gene therapy vectors such as adenoviruses including helper-dependent adenoviral vectors.

Helper-dependent adenoviruses (HDAd), also known as gutless or high-capacity adenoviruses, are the latest generation of adenoviral vectors (Mitani, K., Graham, F. L. Caskey, C. T. & Kochanek, S. Rescue, propagation, and partial purification of a helper virus-dependent adenovirus vector. Proc Natl Acad Sci USA 92, 3854-3858 (1995); Parks, R. J. et al. A Helper-dependent adenovirus vector system: removal of helper virus by Cre-mediated excision of the viral packaging signal. Proc Natl Acad Sci USA 93, 13565-13570 (1996); Parks, R. J. Improvements in adenoviral vector technology: overcoming barriers for gene therapy. Clin. Genet. 58, 1-11 (2000)). These vectors are devoid of all viral sequences and are able to mediate long-term gene expression in various tissues (e.g. 7 years in the liver) in contrast to the more immunogenic first generation adenoviruses (Brunetti-Pierri, N., Ng, T., Iannitti, D., Cioffi, W., Stapleton, G., Law, M., Breinholt, J., Palmer; D., Grove, N., Rice, K., et al. (2013). Transgene Expression up to 7 Years in Nonhuman Primates Following Hepatic Transduction with Helper-Dependent Adenoviral Vectors. Hum Gene Ther 24, 761-765.).

### BRIEF SUMMARY OF THE INVENTION

In one aspect, provided herein is a gene therapy delivery and expression system, comprising at least one helper-dependent adenoviral vector containing a nucleic acid sequence encoding proteoglycan 4 (PRG4), or a biologically active fragment thereof, which has chondoprotective activity, left and right adenoviral inverted terminal repeats (L ITR and R ITR), adenoviral packaging signal sequences and non-viral, non-coding stuffer nucleic acid sequences, wherein PRG4 expression in the at least one helper-dependent adenoviral vector is controlled by a ubiquitous, constitutive promoter, wherein (i) the helper-dependent adenoviral vector additionally comprises a nucleic acid sequence encoding interleukin-1 receptor antagonist (Il-1Ra), or (ii) the delivery and expression system comprises a second helper-dependent adenoviral vector comprising a nucleic acid sequence encoding interleukin-1 receptor antagonist (Il-1Ra).

In some embodiments, the ubiquitous constitutive promoter is selected from the group consisting of elongation factor 1 alpha (EF1 alpha) promoter, cytomegalovirus (CMV) promoter, beta-actin promoter, simian virus 40 (SV40) early promoter, ubiquitin c promoter, glyceraldehyde 3-phosphate dehydrogenase (GAPDH) promoter, phosphoglycerate kinase (PGK) promoter.

In some embodiments, the helper-dependent adenoviral vector containing a nucleic acid sequence encoding proteoglycan 4 (PRG4) comprises a nucleic acid sequence set forth in SEQ ID NO 1, or SEQ ID NO 2, or a biologically active fragment thereof, or a nucleic acid sequence which has at least 50%, 60%, 70%, 80% or 90% sequence homology with a nucleic acid sequence set forth in SEQ ID NO 1, or SEQ ID NO 2, or a biologically active fragment thereof, wherein the helper-dependent adenoviral vector containing a nucleic acid sequence encoding PRG4, which comprises a biologically active fragment of SEQ ID NO 1, or SEQ ID NO 2, or a nucleic acid sequence which has at least 50%, 60%, 70%, 80% or 90% sequence homology with a nucleic acid sequence set forth in SEQ ID NO 1. or SEQ ID NO 2, or a biologically active fragment thereof, mediates chondoprotective activity.

In some embodiments, the nucleic acid sequence encoding proteoglycan 4 (PRG4) comprises a nucleic acid sequence set forth in SEQ ID NO 3, or SEQ ID NO 4, or a biologically active fragment thereof, or a homolog thereof from any other species, or a nucleic acid sequence which has at least 50%, 60%, 70%, 80% or 90% sequence homology with a nucleic acid sequence set forth in SEQ ID NO 3, or SEQ ID NO 4, or a biologically active fragment thereof, wherein the PRG4 encoded by the biologically active fragment of SEQ ID NO 3, or SEQ ID NO 4, or a nucleic acid sequence which has at least 50%, 60%, 70%, 80% or 90% sequence homology with a nucleic acid sequence set forth in SEQ ID NO 3, or SEQ ID NO 4, or a biologically active fragment thereof, has chondoprotective activity.

In some embodiments, the amino acid sequence of proteoglycan 4 (PRG4) comprises an amino acid sequence set forth in SEQ ID NO 5, or SEQ ID NO 6, or a biologically active fragment thereof, or a homolog thereof from any other species, or an amino acid sequence which has at least 50%, 60%, 70%, 80% or 90% sequence homology with an amino acid sequence set forth in SEQ ID NO 5, or SEQ ID NO 6, or a biologically active fragment thereof, wherein the amino acid sequence of PRG4, which comprises a biologically active fragment of the amino acid SEQ ID NO 5, or SEQ ID NO 6, or has at least 50%, 60%, 70%, 80% or 90% sequence homology with an amino acid sequence set forth in SEQ ID NO 5, or SEQ ID NO 6, or a biologically active fragment thereof, has chondoprotective activity.

In some embodiments, expression of interleukin-1 receptor antagonist (Il-1Ra) is controlled by an inflammation-inducible promoter selected from the group consisting of NF-κB promoter, interleukin 6 (Il-6) promoter, interleukin-1 (Il-1) promoter, tumor necrosis factor (TNF) promoter, cyclooxygenase 2 (COX-2) promoter, complement factor 3 (C3) promoter, serum amyloid A3 (SAA3) promoter, macrophage inflammatory protein-1α (MIP-1α) promoter, or hybrid constructs of the above.

In some embodiments, the helper-dependent adenoviral vector comprising the nucleic acid sequence encoding interleukin-1 receptor antagonist (Il-1Ra), comprises a nucleic acid sequence which has at least 50%, 60%, 70%, 80% or 90% sequence homology with a nucleic acid sequence set forth in SEQ ID NO 7, or SEQ ID NO 8, or SEQ ID NO 9, or a biologically active fragment thereof, wherein the helper-dependent adenoviral vector comprising the nucleic acid sequence encoding Il-1Ra, which comprises a nucleic acid sequence which has at least 50%, 60%, 70%, 80% or 90% sequence homology with a nucleic acid sequence set forth in SEQ ID NO 7, or SEQ ID NO 8, or SEQ ID NO 9, or a biologically active fragment thereof, has Il1-Ra activity of inhibiting inflammatory and cartilage destructive mediators.

In some embodiments, the nucleic acid sequence encoding interleukin-1 receptor antagonist (Il-1Ra) comprises a nucleic acid sequence set forth in SEQ ID NO 10, or SEQ ID NO 11, or SEQ ID NO 12, or a biologically active fragment thereof, or wherein the nucleic acid sequence encoding for interleukin-1 receptor antagonist (Il-1Ra) comprises a nucleic acid sequence which has at least 50%, 60%, 70%, 80% or 90% sequence homology with a nucleic acid sequence set forth in SEQ ID NO 10, or SEQ ID NO 11, or SEQ ID NO 12, or a biologically active fragment thereof, wherein the Il-1Ra encoded by the biologically active fragment of SEQ ID NO 10, or SEQ ID NO 11, or SEQ ID NO 12, or the nucleic acid sequence which has at least 50%, 60%, 70%, 80% or 90% sequence homology with a nucleic acid sequence set forth in SEQ ID NO 10, or SEQ ID NO 11, or SEQ ID NO 12, or the biologically active fragment thereof, mediates Il1-Ra activity of inhibiting inflammatory and cartilage destructive mediators.

In some embodiments, the amino acid sequence of interleukin-1 receptor antagonist (Il-1Ra) comprises an amino acid sequence set forth in SEQ ID NO 13, SEQ ID NO 14, SEQ ID NO 15, or a biologically active fragment thereof, which has Il1-Ra activity of inhibiting inflammatory and cartilage destructive mediators.

In another aspect, provided herein is a pharmaceutical composition, comprising a therapeutically effective amount of at least one helper-dependent adenoviral vector containing a nucleic acid sequence encoding proteoglycan 4 (PRG4), or a biologically active fragment thereof, which has chondoprotective activity, left and right adenoviral inverted terminal repeats (L ITR and R ITR), adenoviral packaging signal sequences and non-viral, non-coding stuffer nucleic acid sequences, wherein PRG4 expression in the at least one helper-dependent adenoviral vector is controlled by a ubiquitous, constitutive promoter, wherein (i) the helper-dependent adenoviral vector additionally comprises a nucleic acid sequence encoding interleukin-1 receptor antagonist (Il-1Ra), or (ii) the delivery and expression system comprises a second helper-dependent adenoviral vector comprising a nucleic acid sequence encoding interleukin-1 receptor antagonist (Il-1Ra).

In some embodiments, the ubiquitous constitutive promoter is selected from the group consisting of elongation factor 1 alpha (EF 1 alpha) promoter, cytomegalovirus (CMV) promoter, beta-actin promoter, simian virus 40 (SV40) early promoter, ubiquitin c promoter, glyceraldehyde 3-phosphate dehydrogenase (GAPDH) promoter, phosphoglycerate kinase (PGK) promoter.

In some embodiments, wherein the helper-dependent adenoviral vector containing a nucleic acid sequence encoding proteoglycan 4 (PRG4) comprises a nucleic acid sequence set forth in SEQ ID NO 1, or SEQ ID NO 2, or a biologically active fragment thereof, or a nucleic acid sequence which has at least 50%, 60%, 70%, 80% or 90% sequence homology with a nucleic acid sequence set forth in SEQ ID NO 1, or SEQ ID NO 2, or a biologically active fragment thereof, wherein the helper-dependent adenoviral vector containing a nucleic acid sequence encoding PRG4, which comprises a biologically active fragment of SEQ ID NO 1, or SEQ ID NO 2, or a nucleic acid sequence which has at least 50%, 60%, 70%, 80% or 90% sequence homology with a nucleic acid sequence set forth in SEQ ID NO 1. or SEQ ID NO 2, or a biologically active fragment thereof, mediates chondoprotective activity.

In some embodiments, the nucleic acid sequence encoding proteoglycan 4 (PRG4) comprises a nucleic acid sequence set forth in SEQ ID NO 3, or SEQ ID NO 4, or a biologically active fragment thereof, or a homolog thereof from any other species, or a nucleic acid sequence which has at least 50%, 60%, 70%, 80% or 90% sequence homology with a nucleic acid sequence set forth in SEQ ID NO 3, SEQ ID NO 4, or a biologically active fragment thereof, wherein the PRG4 encoded by the biologically active fragment of SEQ ID NO 3, or SEQ ID NO 4, or a nucleic acid sequence which has at least 50%, 60%, 70%, 80% or 90% sequence homology with a nucleic acid sequence set forth in SEQ ID NO 3, or SEQ ID NO 4, or a biologically active fragment thereof, has chondoprotective activity.

In some embodiments, the amino acid sequence of proteoglycan 4 (PRG4) comprises an amino acid sequence set forth in SEQ ID NO 5, or SEQ ID NO 6, or a biologically active fragment thereof, or a homolog thereof from any other species, or an amino acid sequence which has at least 50%, 60%, 70%, 80% or 90% sequence homology with an amino acid sequence set forth in SEQ ID NO 5, or SEQ ID NO 6, or a biologically active fragment thereof, wherein the amino acid sequence of PRG4, which comprises a biologically active fragment of the amino acid SEQ ID NO 5, or SEQ ID NO 6, or has at least 50%, 60%, 70%, 80% or 90% sequence homology with an amino acid sequence set forth in SEQ ID NO 5, or SEQ ID NO 6, or a biologically active fragment thereof, has chondoprotective activity.

In some embodiments, expression of interleukin-1 receptor antagonist (Il-1Ra) is controlled by an inflammation-inducible promoter selected from the group consisting of NF-κB promoter, interleukin 6 (Il-6) promoter, interleukin-1 (Il-1) promoter, tumor necrosis factor (INF) promoter, cyclooxygenase 2 (COX-2) promoter, complement factor 3 (C3) promoter, serum amyloid A3 (SAA3) promoter, macrophage inflammatory protein-1α (MIP-1α) promoter, or hybrid constructs of the above.

In some embodiments, the helper-dependent adenoviral vector comprising the nucleic acid sequence encoding interleukin-1 receptor antagonist (Il-1Ra), comprises a nucleic acid sequence which has at least 50%, 60%, 70%, 80 or 90% sequence homology with a nucleic acid sequence set forth in SEQ ID NO 7, or SEQ ID NO 8, or SEQ ID NO 9, or a biologically active fragment thereof, wherein the helper-dependent adenoviral vector comprising the nucleic acid sequence encoding Il-1Ra, which comprises a nucleic acid sequence which has at least 50%, 60%, 70%, 80% or 90% sequence homology with a nucleic acid sequence set forth in SEQ ID NO 7, or SEQ ID NO 8, or SEQ ID NO 9, or a biologically active fragment thereof, has Il1-Ra activity of inhibiting inflammatory and cartilage destructive mediators.

In some embodiments, the nucleic acid sequence encoding interleukin-1 receptor antagonist (Il-1Ra) comprises a nucleic acid sequence set forth in SEQ ID NO 10, or SEQ ID NO 11, or SEQ ID NO 12, or a biologically active fragment thereof, or wherein the nucleic acid sequence encoding for interleukin-1 receptor antagonist (Il-1Ra) comprises a nucleic acid sequence which has at least 50%, 60%, 70%, 80% or 90% sequence homology with a nucleic acid sequence set forth in SEQ ID NO 10, or SEQ ID NO 11, or SEQ ID NO 12, or a biologically active fragment thereof, wherein the Il-1Ra encoded by the biologically active fragment of SEQ ID NO 10, or SEQ ID NO 11, or SEQ ID NO 12, or the nucleic acid sequence which has at least 50%, 60%, 70%, 80% or 90% sequence homology with a nucleic acid sequence set forth in SEQ ID NO 10, or SEQ ID NO 11, or SEQ ID NO 12, or the biologically active fragment thereof, mediates Il1-Ra activity of inhibiting inflammatory and cartilage destructive mediators.

In some embodiments, the amino acid sequence of interleukin-1 receptor antagonist (Il-1Ra) comprises an amino acid sequence set forth in SEQ ID NO 13, SEQ ID NO 14, SEQ ID NO 15, or a biologically active fragment thereof, which has Il1-Ra activity of inhibiting inflammatory and cartilage destructive mediators.

In another aspect, provided herein is the gene therapy delivery and expression system according to any of the preceding embodiments for use in the prevention and/or treatment of camptodactyly-arthropathy-coxa vara-pericarditis (CACP) syndrome, or a musculoskeletal disorder, or a joint disorder or disease. In some embodiments, the disease or disorder is selected from the group consisting of arthropathies, all types of arthritis, including arthritis-related disorders, osteoarthritis, rheumatoid arthritis, gout and pseudo-gout, septic arthritis, psoriatic arthritis, ankylosing spondylitis, juvenile idiopathic arthritis, Still's disease, Reiter's syndrome, or tendinopathies including tendonitis, tendinosis, tenosynovitis; synovial disorders including synovitis; Bursa disorders including bursitis; equine musculoskeletal disorders including bone spavin, navicular syndrome, osselet.

### BRIEF DESCRIPTION OF THE FIGURES

FIG. 1. Prg4 transgenic mice are protected from development of age-related osteoarthritis. A, Comparison of 10 month-old wild type mice and Prg4 transgenic mice knee joints by OARSI grade (*P<0.05, n=7. Wilcox rank test). B, Safranin O staining and immunohistochemistry (antibody used to the left) of 10 month-old wild type (Wt) and Prg4 transgenic mice. Black arrows indicate osteoarthritis changes in proximal tibial articular cartilage in sagittal section, Scale bar, 100 pm μm. C, Representative image of the reconstruction of articular cartilage in 10-month-old wild type and Prg4 transgenic mouse with femoral cartilage shown in blue and tibial cartilage shown in yellow. Red arrow indicates loss of cartilage. Scale bar, 500 μm. D, Quantification of articular cartilage volume and surface area of bone covered by cartilage in mouse knee joints by phase contrast μCT. (*P<0.01, n=5, t-test). Error bars indicate s.e.m.

FIG. 2. Prg4 transgenic mice are protected from the development of post-traumatic osteoarthritis, A, Comparison of Prg4 transgenic mice and wild type mice knee joints by OARSI grade (*P<0.05, n=8, ANOVA). B, Safranin O staining and immunohistochemistry (antibody used listed above each column) of wild type sham (Wt Sham), wild type with transection (Wt Sx) and Prg4 transgenic mice with transection (Prg4 Sx). Black arrows indicate areas with osteoarthritis changes in saggital sections through the knee. Scale bar, 100 μm. C, Representative images of reconstruction of knee joints with phase contrast μCT with femoral cartilage shown in blue and tibial cartilage shown in yellow. Red arrow indicates loss of cartilage, Scale bar, 500 μm. D, Quantification of articular cartilage volume and surface area of bone covered by cartilage in mouse joints by phase contrast μCT (*P<0.01, **P<0.05, N.S.=not significant, n=5-6, ANOVA). E, Average time that mice stayed on rotating rod 2 months after cruciate ligament transection in rotarod analysis (*P<0.05, n=15, ANOVA). F, Response time of mice after placement onto a 55° C. platform in hotplate analysis (*P<0.05, n=10, ANOVA), Error bars indicate s.e.m.

FIG. 3: PRG4 delivered by helper-dependent adenoviral vectors (HDAd) protects mice from development of osteoarthritis. A, Representative image comparing intra-articular injection of HDAd and different serotypes of AAV. Vectors (10⁹ viral particles) each expressing GFP were injected intraarticularly into the knee joint. The lower panels are enlarged images of the boxed areas in the upper panels. Green: GFP, blue: DAPI. C: cartilage, S: synovium. Scale bar, 100 μm. B, Comparison of luciferase expression after intra-articular injection of first-generation adenoviral vector (FGV) and helper-dependent adenoviral vector (HDAd) (n=6) into the knee joint. C, Representative image of expression patterns of intra-articular injections of different doses of HDAD encoding □-galactosidase. The bottom images are enlarged areas of the black box in the upper images of saggital sections through the knee joint. C: Cartilage; S: Synovium. Scale bar, 100 μm. D-G, Scheme of experiment comparing the preventive effect HDAd-PRG4 injection and evaluation by OARSI grade and cartilage volume (D). Before transection, wild type mice were injected with HDAd-PRG4 intra-articularly at 10⁹ vp/joint (Sx+PRG4 H) or 10⁸ vp/joint (Sx+PRG4 L). Sham, transection without treatment (Sx) and injection of virus without transgene before transection (Sx+Vector) served as controls. Degree of osteoarthritis is presented by OARSI grade (E), cartilage volume (F) and cartilage surface area (G) (*P<0.05, n=8-10 in OARSI grading, n=5-6 in cartilage volume analysis, ANOVA). H-K, Scheme of experiment comparing the protective effect HDAd-PRG4 injection and evaluation by OARSI grade and cartilage volume (H). Two weeks after transection, wild type mice were injected with HDAd-PRG4 intra-articularly at 10⁹ vp/joint (Sx+PRG4 L) or 10⁸ vp/joint (Sx+PRG4 L), Sham, no treatment (Sx) and HDAd-GFP injection (Sx+GFP) served as controls. Degree of OA is presented by OARSI grade (I), cartilage volume (J) and cartilage surface area (K) (*P<0.05, n=8-10 in OARSI grading, n=5-6 in cartilage volume analysis, ANOVA). Error bars indicate s.e.m.

FIG. 4: PRG4 delays osteoarthritis by inhibiting cartilage catabolism and terminal hypertrophy. A, Microarray heat map analysis comparing superficial zone cartilage of wild type and Prg4 transgenic mice, Genes with expression changes larger than 1.5 fold and p-value less than 0.05 are plotted. B, Transcription factor activity changes predicted by Ingenuity Pathway Analysis. All transcription factors shown here were predicted to be suppressed by Ingenuity Pathway Analysis. X-axis indicates the number of genes/gene groups controlled by each transcription pathway in the gene list submitted. C, Changes in gene expression (Prg4, Hif3a, Vegf, Col10a1 and Mmp13) in C3H10T1/2 cells under hypoxia (1% oxygen for 8 hours). Cells are sham treated, infected with empty HDAd (vector) or with HDAd-PRG4 (PRG4) (*P<0.05, n=3, ANOVA). D, Changes of PRG4 and Hif3alpha expression under normoxia and hypoxia in TC71 Ewing sarcoma cells (*P<0.05, n=3, t-test). E, Changes of gene expression after Hif3alpha knockdown by siRNA (*P<0.05, n=3, ANOVA). F, Proposed model of PRG4 function in prevention of osteoarthritis development. Error bars indicate s.e.m.

FIG. 5: PRG4 over-expression under the Col2al promoter does not adversely affect the development of mice. A, Schematic figure of the Prg4 transgenic mice construct. B, Rib cartilage expression levels of PRG4, Sox9 and Col2al in newborns (P1) PRG4 transgenic and wild type mice (*P<0.05, n=7-8, student t-test). C, Comparison of anti-PRG4 antibody stained femoral head section in wild type and Prg4 transgenic newborn mice. Scale bars, 100 μm. D, Comparison of anti-PRG4 antibody stained knee joints in Prg4 transgenic and wild type 3 month-old mice. The boxed areas to the left (articular area, distal femoral growth plate and proximal tibial growth plate) are enlarged at the right. Scale bars, 100 μm. E, Weight of Prg4 high expresser, low expresser and wild type mice (P<0.05, n=10-11). F, Representative image of BrdU (red) and DAPI (blue) staining in wild type and Prg4 transgenic P3 hind limbs. Staining signals were quantified by image J (P<0.05, n=7). Scale bars, 100 μm. G, Representative image of TUNEL (brown) and methyl green staining in wild type and Prg4 transgenic P3 hind limbs. No positive signals were visible in cartilage but they were present in overlying dermis and skin. Scale bar, 100 μm. Error bars indicate s.e.m.

FIG. 6: Generation and characterization of HDAd-PRG4. A, HDAd-PRG4 band is visible in the last step of cesium chloride ultracentrifugation. The purified virus is the white band in the tube. B, PCR determination of PRG4 expression of HEK293 cells infected with HDAd-PRG4 (PRG4), HDAd-GFP (GFP), and sham treatment (cells). Gapdh is used as loading control. C, Immunofluorescence of HEK293 cells infected with HDAd-PRG4 (PRG4, red) and HDAd-GFP (GFP, green). DAPI (blue) was used as counter-stain.

FIG. 7: Gene expression profiling of wild type vs. Prg4 transgenic superficial layer chondrocytes. A, Laser capture micro-dissection of superficial layer chondrocytes. Left to right: tissue before capturing, captured tissue on HS cap, and remaining tissue on the slide. Scale bar, 100 μm. B, Evaluation of Hif3a expression in superficial layer chondrocytes of wild type vs. Prg4 mice (*Py0.05, n=3, t-test). C, Evaluation of PRG4 expression in ATDC5 and C3H10T1/2 cells under normoxic and hypoxic conditions (*P<0.05, n=3, t-test), Error bars indicate s.e.m.

FIG. 8: A, Mice were treated with HDAd-PRG4 (PRG4), HDAd-Il-1Ra (111Ra) and combination therapy (PRG4+Il1Ra) according to the scheme shown in FIG. 3 H. All three groups showed significantly lower histological score compared to no treatment and placebo treatment, suggesting therapeutic effects to HDAd-PRG4 and HDM-Il-1Ra. The combination therapy showed a trend of being more effective, (ANOVA, N=8-10, *p<0.05); B, C, Mice were treated with HDAd-PRG4 (PRG4), HDAd-Il1-Ra (Il1Ra) and combination therapy according to the scheme shown in FIG. 3 H. Cartilage surface area (B) and cartilage volume (C) were quantified. All three groups showed significantly higher cartilage volume and surface area compared to no treatment and placebo treatment, suggesting therapeutic effects of HDAd-PRG4 and HDAd-Il1Ra. The knees under combination therapy had more preserved cartilage volume and surface area (ANOVA, N=5, *p<0.05).

### DETAILED DESCRIPTION

It is therefore an object of the present invention to provide an improved delivery and expression system that allows for long-term expression of biologically active proteoglycan 4 (PRG4) for use in the prevention and/or treatment of PRG4-dependent disorders such as camptodactyly-arthropathy-coxa vara-pericarditis (CACP) and disorders in which PRG4 overexpression is beneficial such as musculoskeletal disorders in particular joint disorders.

The solution for the problem is provided by a gene therapy delivery and expression system, comprising the technical features as claimed in claim 1. Preferred embodiments of the invention are subject-matter of the dependent claims.

The gene therapy delivery and expression system according to the present invention comprises at least one helper-dependent adenoviral vector containing a nucleic acid sequence encoding for proteoglycan 4 (PRG4), or a biologically active fragment thereof, left and right adenoviral inverted terminal repeats (L ITR and R ITR), adenoviral packaging signal sequences and non-viral, non-coding stuffer nucleic acid sequences.

Any known left or right adenoviral inverted terminal repeats (L ITR and R ITR), adenoviral packaging signal sequences and non-viral, non-coding stuffer nucleic acid sequences can be used for the production of the helper-dependent adenoviral vector (Parks, R. J., Chen, L., Anton, M., Sankar, U., Rudnicki, M. A., and Graham, F. L. (1996). A helper-dependent adenovirus vector system: removal of helper virus by Cre-mediated excision of the viral packaging signal. Proc Natl Acad Sci USA 93, 13565-13570; Palmer, D. and Ng, P. (2003). Improved system for helper-dependent adenoviral vector production. Mol Ther 8, 846-852.).

The results and data shown herein are based on helper-dependent adenoviral constructs (HDAd) using in a first embodiment nucleic acid sequences or amino-acid sequences encoding for proteoglycan 4 (PRG4). Any homolog or variant showing a certain degree of sequence homology with either a nucleic acid sequence or an amino-acid sequence of proteoglycan 4 (PRG4) (or their variant such as lubricin, superficial zone protein or megakaryocyte stimulating factor precursor) is further comprised by the present invention. A homolog includes but is not limited to peptides, polypeptides, proteins or nucleic acid sequences from any species that shows homology with any proteoglycan (PRG4) described herein,

For long-term expression of PRG4 in the affected tissue, for example in joints or osteoarthritic tissues, the at least one helper-dependent adenoviral vector of the invention is preferably controlled by a ubiquitous, constitutive promoter. Suitable promoters include, but are not limited to elongation factor 1 alpha (EF1 alpha) promoter, cytomegalovirus (CMV) promoter, beta-actin promoter, simian virus 40 (SV40) early promoter, ubiquitin c promoter, glyceraldehyde 3-phosphate dehydrogenase (GAPDH) promoter, phosphoglycerate kinase (PGK) promoter and other HDAd-suitable ubiquitous, constitutive promoters.

In a preferred embodiment, the helper-dependent adenoviral vector comprising proteoglycan 4 (PRG4) comprises a nucleic acid sequence set forth in SEQ ID NO 1 (human HDAd) or SEQ ID NO 2 (murine HDAd). Preferably, the nucleic acid sequence comprises a cDNA sequence of the PRG4 gene or a fragment thereof. Furthermore, any biologically active fragment such as nucleic acid sequences having sequence identity or a certain degree of homology with the human or murine PRG4 sequence disclosed herein is comprised by the present invention. The HDAd of the invention may vary in its non-coding elements as well as in the length of its coding insert. Therefore, also smaller or greater vector sizes of the helper-dependent adenoviral vector of the invention can be utilized for the purpose of the present invention in order to achieve the desired biological effects.

In a preferred embodiment, the helper-dependent adenoviral vector comprises a nucleic acid sequence which has at least 50%, 60%, 70%, 80% or 90% sequence homology with a vector sequence comprising a nucleic acid sequence set forth in SEQ ID NO 1 or SEQ ID NO 2, or a biologically effective fragment thereof.

A sequence homology of at least 50% with a nucleic acid sequence set forth in SEQ ID NO 1 or SEQ ID NO 2 can be sufficient in order to generate long-term expression of PRG4 at the target sites as along as the expressed PRG4 protein is biologically active.

In one embodiment the helper-dependent adenoviral vector comprises a nucleic sequence encoding for proteoglycan 4 (PRG4). The expression of human or mammalian PRG4 is preferred. The inserted nucleic acid sequence into the HDAd can be anyone, which shows sequence identity or sequence homology with a nucleic acid sequence set forth in SEQ ID NO 3 or SEQ ID NO 4. For the purpose of expression, it can be sufficient that only a part of the nucleic acid sequence set forth in SEC ID NO 3 or SEQ ID NO 4, or an extended version is sufficient for the generation of the vector and its biological activity. The biological activity can be measured by investigating chondoprotection.

Furthermore, also mutants, variants and homologs containing nucleic acid replacements within the amino acid or nucleic acid sequence of proteoglycan 4 (PRG4) are comprised by the present invention. In particular, the invention comprises a homolog of PRG4 from any other animal species having sequence homology with a sequence set forth in SEQ ID NO 3 or SEQ ID NO 4. For example, a homolog containing a nucleic acid sequence encoding for proteoglycan 4 (PRG4) preferably comprises a nucleic acid sequence, which has at least 50%, 60%, 70%, 80% or 90% sequence homology with a nucleic acid sequence set forth in SEQ ID NO 3, or SEQ ID NO 4, or a biologically effective fragment thereof.

The proteoglycan 4 (PRG4) used in the construction of the vector of the present invention can be also described by its amino acid sequence. Preferably, the proteoglycan 4 (PRG4) comprises an amino acid sequence set forth in SEQ ID NO 5 or SEQ ID NO 6, or a biologically active fragment thereof, or a homolog thereof from any other species. In a preferred embodiment, the amino acid sequence encoding for proteoglycan 4 (PRG4) comprises an amino acid sequence which has at least 50%, 60%, 70%, 80% or 90% sequence homology with an amino acid sequence set forth in SEQ ID NO 5, or SEQ ID NO 6, or a biologically active fragment thereof, or a homolog thereof from any other species.

The inventors show herein that a helper-dependent adenoviral vector containing the cDNA sequence of murine or human proteoglycan 4 (PRG4) under the control of a ubiquitous, constitutive promoter results in an overexpression of PRG4 in joints, which results in protection of mice from osteoarthritis. Since overexpression of PRG4 can be beneficial in many other diseases, the helper-dependent vector of the invention can be used for the manufacture of a medicament for the treatment and/or prevention of a variety musculoskeletal diseases and PRG4-dependent diseases. In particular, musculoskeletal disorders, which benefit from PRG4 over-expression would be characterised as those disorders where high concentrations of PRG4 confer a therapeutic or preventive effect. PRG4-dependent diseases or disorders would be characterised in that natural PRG4 expression is limited or inhibited, or in that intracellular or extracellular PRG4 RNA or protein levels are significantly reduced.

The inventors compared joint transduction efficiency of helper-dependent adenoviral vectors with different AAV serotype that had been reported by others to be useful in joint gene therapy approaches. Surprisingly, the inventors found that helper-dependent adenoviral vectors showed superior transduction compared with all tested AAV serotypes resulting in transduction of synoviocytes and chondrocytes. The inventors further showed that helper-dependent adenoviral vectors allow for transgene expression that persists for an extended period, whereby the need for repeated administrations of the medicament to an object suffering from a disease in which overexpression of PRG4 may be beneficial will be greatly reduced. In a model of osteoarthritis in mice, the inventors demonstrated that treatment of osteoarthritic joints with the HDAd-PRG4 results in lower osteoarthritis histology scores, indicating that PRG4 exhibits its biological effect by protecting the subject from post-traumatic osteoarthritis. Protection against osteoarthritis with HDAd-PRG4 was demonstrated using two different schemes. In the first scheme mice were injected with HDAd-PRG4 before osteoarthritis was induced. The results of this experiment show that HDAd-PRG4 can be used in the prevention of osteoarthritis. In the second scheme, osteoarthritis was induced before HDAd-PRG4 was injected. The results of this experiment demonstrate that HDAd-PRG4 can be used in the treatment of (pre-existing) osteoarthritis. In support of the biological effects of PRG4 in joints, experiments using Prg4-transgenic mice revealed a protection of the subjects against the development of osteoarthritis without other bone phenotypes.

Thus, overexpression of PRG4 under the control of a suitable ubiquitous, constitutive promoter in a HDAd allows for prevention and/or treatment of a variety of musculoskeletal diseases such as arthropathies, all types of arthritis, including arthritis-related disorders, osteoarthritis, rheumatoid arthritis, gout and pseudo-gout, septic arthritis, psoriatic arthritis, ankylosing spondylitis, juvenile idiopathic arthritis. Still's disease, Reiter's syndrome, or tendinopathies including tendonitis, tendinosis, tenosynovitis; synovial disorders including synovitis; Bursa disorders including bursitis; equine musculoskeletal disorders including bone spavin, navicular syndrome, osselet.

Surprisingly, the inventors further found that a combined expression of PRG4 as induced by HDAd-PRG4 and inhibitors of inflammatory and cartilage destructive mediators results in beneficial therapeutic and protective effects. In particular, a combination of expressing of PRG4 and expressing interleukin-1 receptor antagonist (Il-1Ra) resulted in an increased efficiency in the treatment and/or prevention of musculoskeletal disorders.

In a preferred embodiment, the helper-dependent adenoviral vector therefore comprises a nucleic acid sequence encoding for inhibitors of inflammatory and cartilage destructive mediators such as cytokines including Il-1, TNFa, Il-6, 11-7 Il-8, Il-11, Il-15, Il-17, Il-18, Il-21, leukemia inhibitory factor (LIF), oncostatin M; matrix metalloproteases including MMP-1,3,9,13; aggrecanases including ADAMTS-1,4,5; toll-like receptors (TLR) such as TLR2, TLR4; and nuclear factor ‘kappa-light-chain-enhancer’ of activated B-cells (NF-κB).

In a preferred embodiment, the inhibitor of inflammatory and cartilage destructive mediator is interleukin-1 receptor antagonist (Il-1Ra). A combination of overexpression of PRG4 and Il-1Ra is beneficial for the treatment and/or prevention of the diseases mentioned therein. In a first embodiment, the cDNA sequence of interleukin-1 receptor antagonist can be contained in the same helper-dependent adenoviral vector, which contains the cDNA sequence encoding for proteoglycan 4 (PRG4). In a further embodiment, the cDNA sequence of interleukin-1 receptor antagonist can be contained in a second helper-dependent adenoviral vector, which only contains the cDNA sequence encoding for Il-1Ra.

The delivery and expression system of the invention can therefore comprise a second or further helper-dependent adenoviral vector comprising a nucleic acid sequence encoding for inhibitors of inflammatory and cartilage destructive mediators such as interleukin-1 receptor antagonist (Il-1Ra).

In a preferred embodiment, the cDNA of the inhibitor of inflammatory and cartilage destructive mediator (e.g. Il1-Ra cDNA) inserted into HDAd is controlled by an inflammation-inducible promoter. Preferred promoters include, but are not limited to promoters selected from the group consisting of NF-κB promoter, interleukin 6 (Il-6) promoter, interleukin-1 (Il-1) promoter, tumor necrosis factor (TNF) promoter, cyclooxygenase 2 (COX-2) promoter, complement factor 3 (C3) promoter, serum amyloid A3 (SAA3) promoter, macrophage inflammatory protein-1a (MIP-1α) promoter, or hybrid constructs of the above. The use of NF-κB promoter in HDAd for the purpose of an inflammation-dependent expression of Il-1Ra at the target sites is preferred.

In a preferred embodiment, the helper-dependent adenoviral vector containing the interleukin-1 receptor antagonist (Il-1Ra) comprises a nucleic acid sequence which has at least 50%, 60%, 70%, 80% or 90% sequence homology with a nucleic acid sequence set forth in SEQ ID NO 7 (human Il-1Ra), or SEQ ID NO 8 (murine Il-1Ra), SEQ ID NO 9 (equine Il-1Ra), or a biologically effective fragment thereof.

In a further embodiment, the nucleic acid sequence encoding for interleukin-1 receptor antagonist (Il-1Ra) comprises a nucleic acid sequence set forth in SEQ ID NO 10 (human Il-1Ra), or SEQ ID NO 11 (murine Il-1Ra), or SEQ ID NO 12 (equine Il-1Ra), or a biologically active fragment thereof, or a homolog thereof from any other species.

In a preferred embodiment, the amino acid sequence encoding for interleukin-1 receptor antagonist (Il-1Ra) comprises an amino acid sequence set forth in SEQ ID NO 13 (human Il-1Ra), or SEQ ID NO 14 (murine Il-1Ra), or SEQ ID NO 15 (equine Il-1Ra), or a biologically active fragment thereof, or a homolog thereof from any other species.

The present invention also relates to a pharmaceutical composition, comprising a therapeutically effective amount of at least one helper-dependent adenoviral vector containing a nucleic acid sequence encoding for proteoglycan 4 (PRG4), or a biologically active fragment thereof. Preferred embodiments of the pharmaceutical composition comprise helper-dependent adenoviral vectors or a combination of different helper-dependent adenoviral vectors comprising features as described above in more detail.

The gene therapy delivery and expression system according to the present invention is suitable for the preparation of a medicament for the use in the prevention and/or treatment of a variety of PRG4-dependent diseases. In a first embodiment the gene therapy delivery and expression system is used in the treatment and/or prevention of camptodactyly-arthropathy-coxa vara-pericarditis (CACP). The helper-dependent adenoviral vectors of the invention can further be used in the prevention and/or treatment of a musculoskeletal disorder, in particular the prevention and/or treatment of a joint disorder or disease. Examples of such diseases are arthropathies, all types of arthritis, including arthritis-related disorders, osteoarthritis, rheumatoid arthritis, gout and pseudo-gout, septic arthritis, psoriatic arthritis, ankylosing spondylitis, juvenile idiopathic arthritis, Still's disease, Reiter's syndrome, or tendinopathies including tendonitis, tendinosis, tenosynovitis; synovial disorders including synovitis; Bursa disorders including bursitis; equine musculoskeletal disorders including bone spavin, navicular syndrome, osselet.

The following examples show the beneficial therapeutic uses of the gene therapy delivery and expression system according to the present invention. In particular, it will be shown that intra-articular expression of proteoglycan 4 (PRG4) in mice protects against development of osteoarthritis (OA). The data are supported by long-term PRG4 expression under the type II collagen promoter (Col2a1) in transgenic mice. Accordingly long-term expression of PRG4 does not adversely affect skeletal development but protects from developing signs of age-related osteoarthritis.

The protective effect is also shown in a model of post-traumatic osteoarthritis created by cruciate ligament transection (CLT). Moreover, intra-articular injection of helper-dependent adenoviral virus (HDAd) expressing PRG4 protected against the development of post-traumatic osteoarthritis when administered either before or after injury. Gene expression profiling of mouse articular cartilage and in vitro cell studies show that PRG4 expression inhibits the transcriptional programs that promote cartilage catabolism and hypertrophy through the up-regulation of hypoxia inducible factor 3 alpha. Analyses of available human osteoarthritis datasets are consistent with the predictions of this model. Hence, the data provide insight into the mechanisms for osteoarthritis development and offer a potential chondroprotective approach to its treatment.

Moreover, injection of helper-dependent adenoviral vectors expressing PRG4 (HDAd-PRG4) and Il-1Ra (HDAd-Il1-Ra) in combination into joints of wild type mice after transection of cruciate ligaments exhibited protective effects against osteoarthritis. Co-injection of HDAd-PRG4 and HDAd-Il1-Ra at the same dose results in a greater extent of cartilage preservation compared to single vector injections.

As such PRG4 in single application or in combination with Il-1Ra is a novel target in chondoprotection using the helper-dependent adenoviral vectors of the invention.

### Examples

Results

PRG4 Prevents Development of Age Related Osteoarthritis Changes

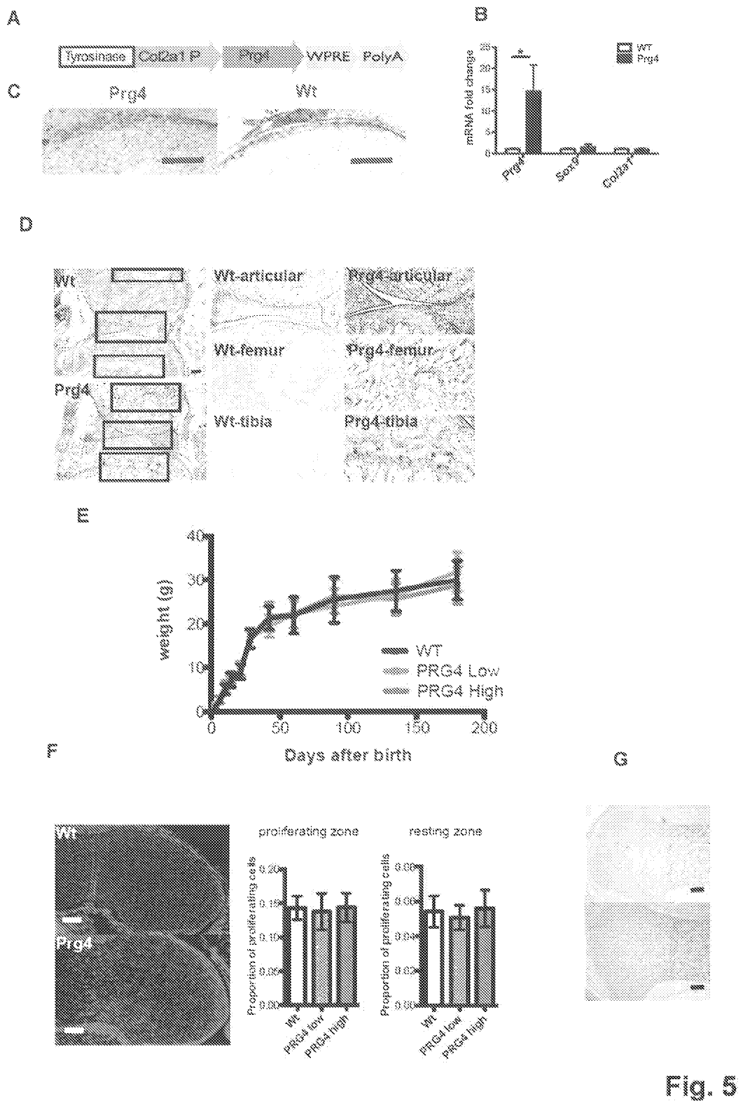

To investigate the long-term effect of Prg4 over-expression, the inventors generated transgenic mice expressing Prg4 under the cartilage specific type II collagen promoter (Col2a1) (FIG. 5A). PRG4 transgenic mice expressed Prg4 ectopically in growth plate cartilage and over-express Prg4 in articular cartilage throughout development and adulthood (FIG. 5B-D). Macroscopically, the inventors detected no differences in growth or skeletal development in Prg4 transgenic mice when assessed by weight (FIG. 5E). Microscopically, markers of chondrocyte proliferation or apoptosis remained the same in Prg4 mice vs. wild type mice as assessed by BrdU staining (p=n.s. in both proliferating and resting zone of P1 chondrocytes) (FIG. 5F) and TUNEL staining (no positive signals in P1 chondrocytes) (FIG. 5G), respectively. These data suggested that ectopic over-expression of Prg4 in cartilage did not significantly affect chondrocyte or skeletal homeostasis.

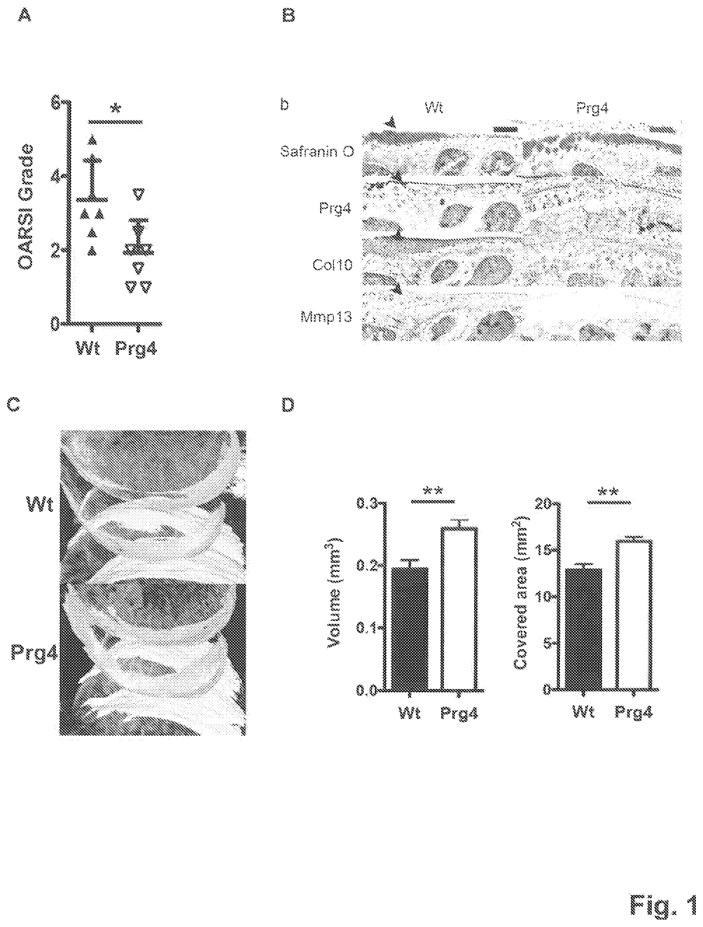

The inventors sought to determine whether Prg4 over-expression in particular chondrocytes protected mice from age-related osteoarthritic changes. Relatively few studies have been performed to assess the development of age-related osteoarthritis in animal models (M. Silbermann, E. Livne, Age-related degenerative changes in the mouse mandibular joint. Journal of Anatomy 129, 507 (October, 1979). Moreover, no gain of function model has been shown to be protective against age-related osteoarthritis. In an aging cohort, as assessed by the Osteoarthritis Research Society International (OARSI) histological grading scale (S. S. Glasson, M. G. Chambers, W. B. van den Berg, C. B. Little, The OARSI histopathology initiative—recommendations for histological assessments of osteoarthritis in the mouse. Osteoarthritis and cartilage/OARSI, Osteoarthritis Research Society 18, S17 (Oct. 1, 2010), the inventors observed that wild type FVB/N mice developed changes consistent with moderate osteoarthritis by 10 months of age, with a mean OARSI grade of 3.5. However, PRG4 transgenic mice at the same age exhibited a mean OARSI grade of 2 (p<0.05), suggesting less severe signs of osteoarthritis (FIG. 1A). The inventors next assessed the molecular differences in particular chondrocytes between the PRG4 and wild type mice. As Collagen type X (Col10a1) and matrix metalloproteinase 13 (Mmp13) are markers of cartilage hypertrophy and degradation, respectively, increased expression of Mmp13 and Col10a1 above the tide marks are hallmarks of osteoarthritis (T. Saito et al. Transcriptional regulation of endochondral ossification by HIF-2α during skeletal growth and osteoarthritis development. Nature Medicine 16, 678 (Jun. 23, 2010)). Encouragingly, the inventors detected increased expression of Col10a1 and Mmp13 in wild type mice with aging, while Prg4 transgenic mice did not show a qualitative increase in either marker in the noncalcified region of articular cartilage (FIG. 1b). These results suggest that Prg4 overexpression had a protective effect against osteoarthritis at the molecular and histological levels.

A disadvantage of conventional histological endpoints is the lack of three-dimensional quantification as well as ascertainment bias based on choice of sections. Hence, the inventors applied an approach to quantify cartilage properties (e.g., volume, surface area, bone area covered by cartilage) based on three-dimensional reconstructions of phase contrast μCT imaging data (M. Ruan et al., Quantitative imaging of murine osteoarthritic cartilage by phase contrast micro-computed tomography. Arthritis Rheum, (2012)). Using this imaging technique, the inventors found that wild type mice showed a decrease in articular cartilage volume as well as in the bone area covered by cartilage (FIG. 1C,D). In contrast, Prg4 transgenic mice showed preservation of articular cartilage volumes and surface area (p<0.01) (FIG. 1C,D). Thus, as compared to wild type mice after transection, Prg4 transgenic mice had none of the histological, molecular and imaging findings characteristic of osteoarthritis. These data suggested that PRG4 over-expression may have protective effects in the context of age-related osteoarthritic-like changes.

PRG4 Prevents Development of Post-Traumatic Osteoarthritis

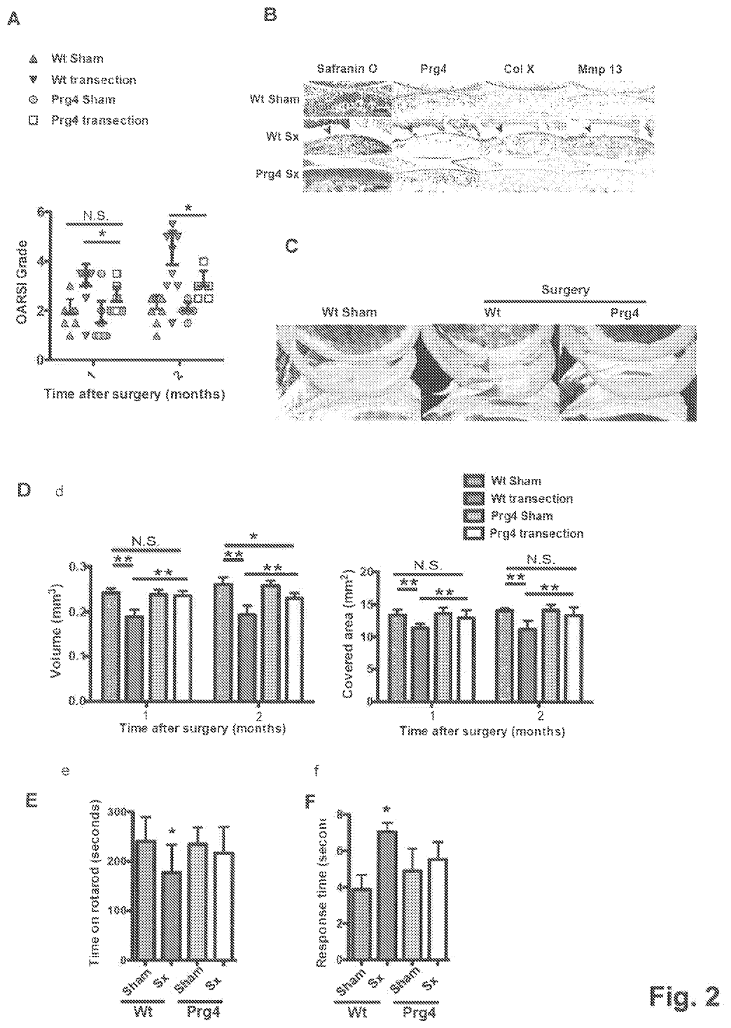

To test whether PRG4 over-expression protects mice from the development of more aggressive, post-traumatic osteoarthritis, the inventors applied the knee cruciate ligament transection model recently developed in the inventors' lab, to both wild type and Prg4 transgenic mice (M. Ruan et al., Quantitative imaging of murine osteoarthritic cartilage by phase contrast micro-computed tomography. Arthritis Rheum, (2012). The inventors chose this approach because anterior cruciate ligament tears are a common cause of post-traumatic arthritis in humans. As assessed by the OARSI histological grading scale, wild type mice developed moderate and severe osteoarthritis one and two months after transection, respectively (FIG. 2A) (M. Ruan et al., Quantitative imaging of murine osteoarthritic cartilage by phase contrast micro-computed tomography. Arthritis Rheum, (2012). Prg4 transgenic mice showed a lower grade OARSI score compared to wild type mice one and two months after transection (FIG. 2A), Interestingly, one month after transection, the OARSI grade of cartilage from Prg4 transgenic mice was not significantly different from wild type mice after sham surgery (FIG. 2A), further supporting that PRG4 expression had a protective effect against osteoarthritis. In addition, the inventors detected increased expression of Col10a1 and Mmp13 in the noncalcified articular cartilage of wild type transected mice, but not in transected Prg4 transgenic mice or wild type mice after sham surgery (FIG. 2B).

The inventors next assessed the cartilage volume and bone area covered by cartilage after surgical transection using phase-contrast microCT (M. Ruan et al., Quantitative imaging of murine osteoarthritic cartilage by phase contrast micro-computed tomography. Arthritis Rheum, (2012)). After transection, wild type mice showed decrease in both cartilage volume and bone area covered by cartilage (p<0.01). In contrast, Prg4 transgenic mice showed articular cartilage volumes and areas similar to wild type mice after sham surgery (FIG. 2C,D). Thus, as compared to wild type mice after transection, Prg4 transgenic mice had none of the histological, molecular or imaging findings characteristic of osteoarthritis.

Pain and motor dysfunction are also hallmarks of osteoarthritis and are typical causes of chronic disability (M. B. Goldling, S. R. Goldring, Osteoarthritis. Journal of Cellular Physiology 213, 626 (2007)), They also serve as important clinical end points for interventional trials. Therefore, the inventors applied rodent behavioral testing, i.e., rotarod and hotplate analyses, to evaluate for potential motor and/or sensory dysfunction in wild type vs. Prg4 transgenic mice after osteoarthritis induction. Surgically transected wild type mice showed a decreased time on the rotarod (p<0.05) and increased time on the hotplate (p<0.05), while Prg4 transgenic mice with and without transection were indistinguishable from wild type mice after sham surgery (p=n.s.) (FIG. 2E,F). This suggested that Prg4 transgenic mice after transection had less motor or sensory impairments as compared to wild type mice, supporting that PRG4 prevented functional impairment in post-traumatic osteoarthritis.

Gene Transfer with HDAd-PRG4 Effectively Treats Osteoarthritis

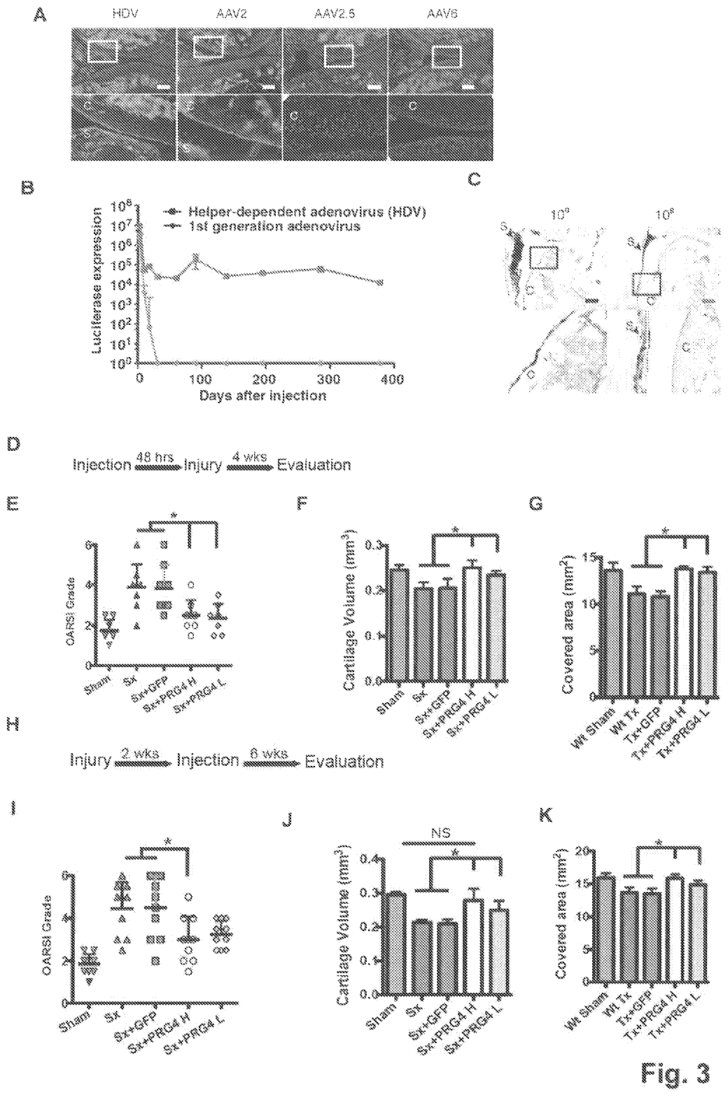

To translate localized expression of PRG4 into a therapeutic approach, the inventors tested whether gene transfer into the joint could mediate long-term expression and chondroprotection in osteoarthritis. Since delivery of recombinant protein is often therapeutically limited by their short half-life, the inventors chose to use a viral gene transfer approach. The most studied viral vectors for gene transfer related to osteoarthritis treatment are adeno-associated virus (AAV) and adenovirus. Both have been shown to transduce chondrocytes in vitro in primary chondrocyte and cartilage organ cultures and in vivo in rabbit and rat knee joints (J. D. Kay et al., Intra-articular gene delivery and expression of interleukin-1 Ra mediated by self-complementary adeno-associated virus. The journal of gene medicine 11, 605 (July, 2009); Y. Arai et al., Gene delivery to human chondrocytes by an adeno associated virus vector. journal of Rheumatol 27, 979 (April, 2000); J. Gouze, Adenovirus-mediated gene transfer of glutamine: fructose-6-phosphate amidotransferase antagonizes the effects of interleukin-1β on rat chondrocytes. Osteoarthritis and Cartilage 12, 217 (April, 2004)). However, no direct comparison has been made between the two viruses. After injection of GFP expressing helper-dependent adenovirus and AAVs of the serotypes 2; 2.5 and 6 into mouse knee joints (10⁹ viral particles per joint in 5 ul), helper-dependent adenovirus was noted to exhibit higher transduction efficiency at 2 weeks post-injection (FIG. 3A).

While first generation adenovirus vectors (FGV) can mediate highly efficient tissue transduction, the immune response to viral proteins limits transgene expression. Previous studies performed by the inventors and others showed that helper-dependent adenoviral vectors (HDAd) devoid of viral coding genes could overcome this problem (D. J. Palmer, D. J. P. D. P. Ng, Helper-dependent adenoviral vectors for gene therapy. Human gene therapy 16, 1 (2005)). For example, a single injection of HDAd can mediate long-term transgene expression in small and large animal models for over 7 years in liver (N. Brunetti-Pierri, P. Ng, Helper-dependent adenoviral vectors for liver-directed gene therapy. Hum Mol Genet 20, R7 (Jun. 13, 2011)). Thus, the inventors tested whether HDAd could mediate long-term expression of luciferase in mouse joint compared to FGVs. Indeed, the inventors found that after a single infra-articular injection, HDAds mediated expression of luciferase in mouse knee joints for over one year, while FGV-mediated luciferase expression was lost by one month (FIG. 3B). To evaluate the dose response and cellular distribution of transduction, the inventors assessed mouse knee joints injected with 10⁹ vs. 10⁸ viral particles HDAd expressing beta-galactosidase. These doses were at least 10 and 100 times lower than the maximum tolerated systemic dose in humans (K. Relph, K. Harrington, H. Pandha, Recent developments and current status of gene therapy using viral vectors in the United Kingdom. BMJ (Clinical research ed.) 329, 839 (Oct. 9, 2004)).

At the higher dose, HDAd transduced superficial layer chondrocytes and synoviocytes, while only synoviocytes were transduced at the lower dose (FIG. 3C). Thus, the inventors showed that HDAd was able to efficiently transduce synoviocytes and chondrocytes with maintaining transgene expression for at least one year.

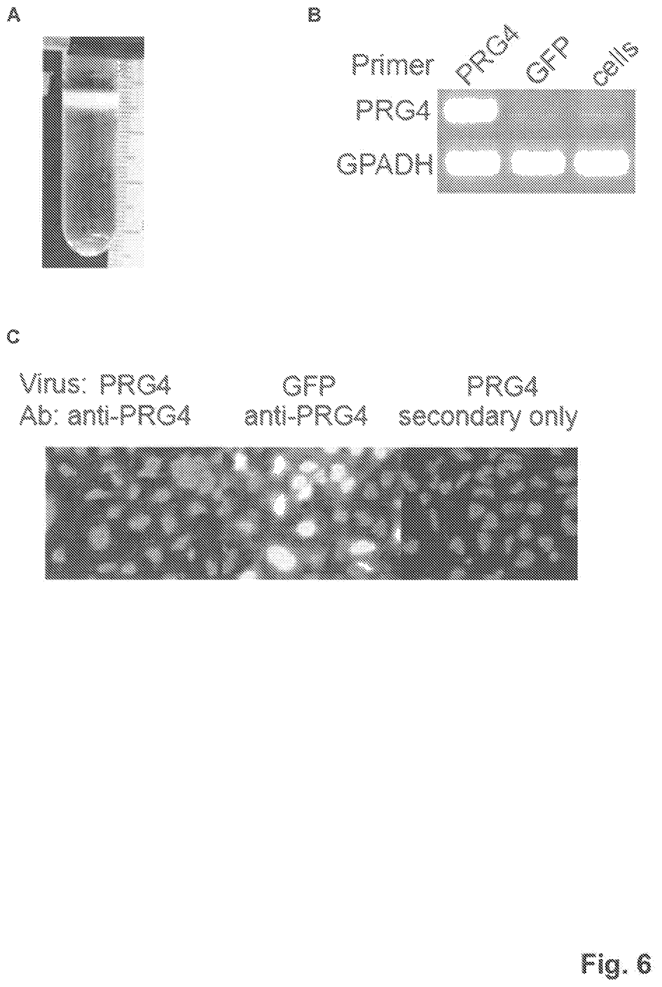

To compare the effects of PRG4 expression from superficial layer chondrocytes vs. synoviocytes, the inventors treated mice at both doses with HDAd expressing PRG4 (FIG. 6A-C) 48 hours prior to surgical cruciate ligament transection. The inventors found that both low dose (mediating expression only in synoviocytes) and high dose (mediating expression in both synoviocytes and superficial zone chondrocytes) treatment with HDAd-PRG4 vector protected joints from osteoarthritis development (FIG. 3D-G). Since the clinical application of PRG4 for osteoarthritis would likely be administrated after an injury, the inventors next tested the efficacy of HDAd-PRG4 injection two weeks after osteoarthritis induction. In this context, injection of the lower dose of HDAd-PRG4 was sufficient to preserve cartilage volumes and prevent cartilage degradation as assessed by μCT, while higher dose injection showed protective effects both by histological OARSI grading and μCT assessment. Importantly, these data suggested that ectopic expression from synoviocytes was sufficient to achieve a certain degree of chondroprotection by acting in a non-cell-autonomous fashion (FIG. 3H-K).

PRG4 Inhibits Transcriptional Programs of Chondrocyte Hypertrophy and Hypoxic Inducible Factors in Cartilage

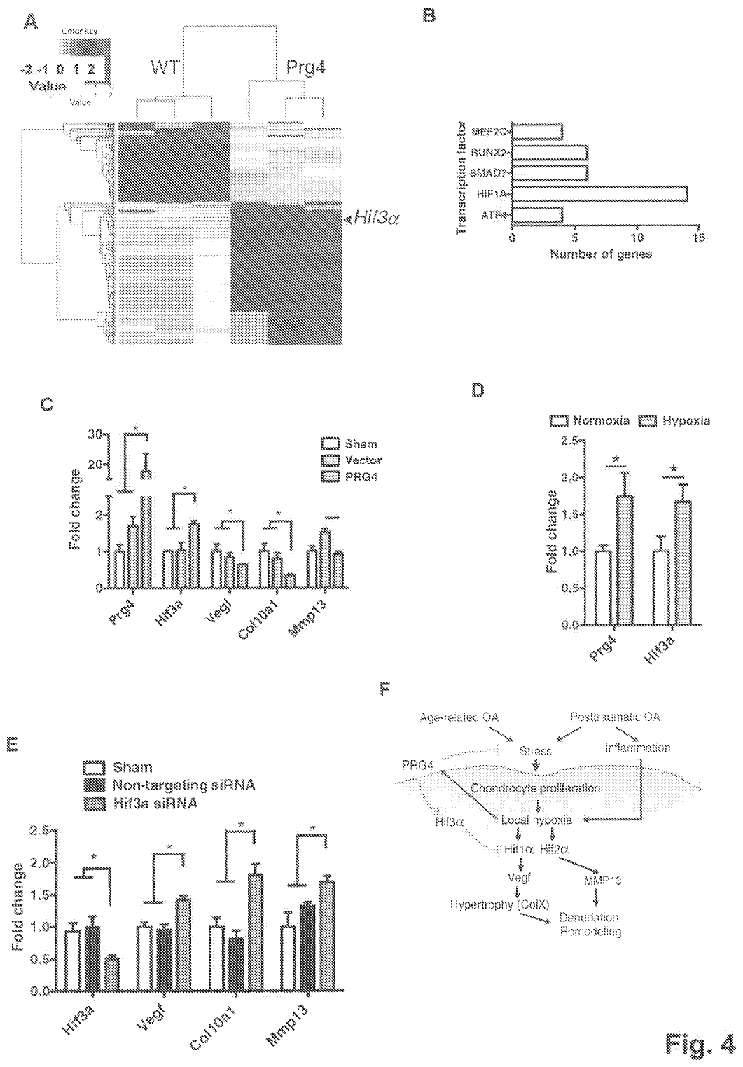

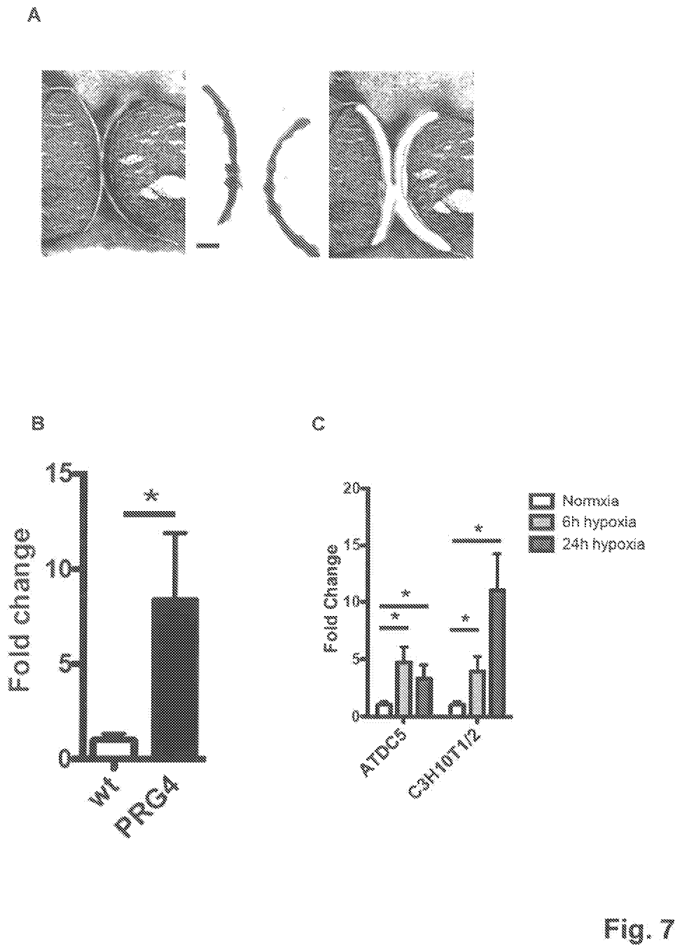

The potential mechanisms of the protective effects of PRG4 have only been partially deciphered. While previous studies have shown that PRG4 relieves mechanical stress in joints by changing synovial fluid dynamics and providing boundary lubrication (G. D. Jay, J. R, Torres, M. L. Warman, M. C. Laderer, K. S. Breuer, The role of lubricin in the mechanical behavior of synovial fluid. Proc Natl Acad Sci USA 104, 6194 (Apr. 10, 2007)), the inventors investigated whether PRG4 could directly affect cartilage metabolism and homeostasis. To assess the molecular effects of PRG4 on chondrocytes, the inventors performed transcriptional profiling on superficial layer chondrocytes obtained by laser capture in newborn wild type vs. Prg4 transgenic mice (FIG. 4A and FIG. 7A). Genes that were either up-regulated or repressed by greater than 1.5 fold (Dataset 1) were analyzed by Ingenuity Pathway Analysis to identify transcriptional programs that would be affected by Prg4 expression (Dataset 2). Interestingly, transcription factors that mediate chondrocyte hypertrophy and terminal differentiation (e.g. Mef2c, Runx2 and Atf4) showed decreased activity with PRG4 over-expression (K. S. Lee et al., Runx2 Is a Common Target of Transforming Growth Factor beta 1 and Bone Morphogenetic Protein 2, and Cooperation between Runx2 and Smad5 Induces Osteoblast-Specific Gene Expression in the Pluripotent Mesenchymal Precursor Cell Line C2C12. Molecular and cellular biology 20, 8783 (Dec. 1, 2000); X. Yang et al., ATF4 is a substrate of RSK2 and an essential regulator of osteoblast biology; implication for Coffin-Lowry Syndrome. Cell 117, 387 (May 2004); M. A. Arnold et al., MEF2C Transcription Factor Controls Chondrocyte Hypertrophy and Bone Development. Developmental Cell 12, 377 (April, 2007)). In addition, the inventors also noted down-regulation of Smad7, an inhibitor of the TGFbeta signaling pathway (A. Nakao et al., Identification of Smad7, a TGFbeta-inducible antagonist of TGF-beta signalling. Nature 389, 631 (Oct. 9, 1997)). TGFbeta signaling negatively regulates terminal differentiation of chondrocytes, and hence. PRG4 again would suppress hypertrophy by its actions on Smad7 (X. Yang et al., TGF-beta/Smad3 signals repress chondrocyte hypertrophic differentiation and are required for maintaining articular cartilage. The Journal of cell biology 153, 35 (May 2, 2001)). Finally, Hypoxia inducible factor 1 alpha unit (Hif1alpha), an essential transcription factor induced in response to hypoxia, was also predicted to have lower activity (FIG. 4B), while Hif3alpha a post-translational negative regulator of Hif1alpha and Hif2alpha was up-regulated (Y. Makino et al., Inhibitory PAS domain protein is a negative regulator of hypoxia-inducible gene expression. Nature 414, 550 (Nov. 29, 2001)). (FIG. 4A and FIG. 7B).

The inventors hypothesized that PRG4 could up-regulate Hif3alpha under hypoxic conditions to inhibit cartilage turnover. This effect would be mediated by down-regulating the Hif1alpha and Hif2alpha transcriptional activities. To test our hypothesis, the inventors measured Hif3alpha expression and downstream Hif target genes relevant to osteoarthritis progression under hypoxic conditions in C3H10T1/2 (mesenchymal stromal) cells. After injection of HDAd-PRG4, Hif3alpha was transcriptionally up-regulated while Vegf, Col101a1 and Mmp13, all markers of hypertrophy, were all down-regulated compared to empty vector (FIG. 4C) (T. Saito et al., Transcriptional regulation of endochondral ossification by HIF-2α during skeletal growth and osteoarthritis development. Nature Medicine 16, 678 (Jun. 23, 2010)). Next, the inventors assessed whether knock down of Hif3alpha suppressed the effects caused by over-expression of PRG4. Since the expression of Hif3alpha is low in C3H10T1/2 cells, the inventors tested its expression in alternative chondrogenic lines. Interestingly, compared to under normoxic conditions, all cell lines tested showed increased PRG4 expression under hypoxic conditions (FIG. 4D and FIG. 7C). Consistent with this, Genomatix analysis identified a hypoxia response element in the promoter region of PRG4. The inventors found that Hif3alpha was highly expressed in TC71 Ewing sarcoma cells and further increased with up-regulation of PRG4 under hypoxic conditions (FIG. 4D). As predicted by our previous transcriptomic analysis, knockdown of Hif3alpha in TC71 cells led to down regulation of Hif1alpha and Hif2alpha target genes in the face of PRG4 up regulation (FIG. 4E). Our findings suggested that the secreted protein PRG4 could modify the balance of anabolic and catabolic programs in the context of osteoarthritis pathogenesis.

To investigate whether the signalling pathway discovered in mouse is conserved in humans, the inventors performed in silico analysis on gene expression profiling performed in human osteoarthritis patient samples available from the GEO database (S, Koelling et al., Migratory Chondrogenic Progenitor Cells from Repair Tissue during the Later Stages of Human Osteoarthritis, Stem Cell 4, 324 (May 3, 2009); T, Dehne, C. Karlsson, J. Ringe, M. Sittinger, A. Lindahl, Chondrogenic differentiation potential of osteoarthritic chondrocytes and their possible use in matrix-associated autologous chondrocyte transplantation. Arthritis research & amp; therapy 11, R133 (2009)). The inventors discovered PRG4 and the proposed downstream effector, HIF3alpha, are upregulated in chondrocyte progenitor cells in OA patients by 2.6 fold (p<0.05) and 1.5 fold (p<0.01) respectively. In an independent array set comparing 3 dimensional cultured chondrocytes from osteoarthritis and healthy donors, the inventors observed a similar trend: PRG4 was upregulated by 1.4 fold (p<0.05) and HIF3alpha upregulated by 1.3 fold (p<0.05). In the context of osteoarthritis development, PRG4 and H1F3alpha may both be upregulated as a repair response. In contrast to the sustained over expression of PRG4 in our therapeutic models, this normal response in humans may be insufficient to prevent disease progression.

These data together showed that under the hypoxic conditions of cartilage, PRG4 over-expression may prevent osteoarthritis progression not only by exerting biomechanical effects on the synovial fluid and cartilage interface, but also by regulating the transcriptional networks that specify chondrocyte hypertrophy and catabolism. Cartilage turnover mediated by Hif1alpha and Hif2alpha was inhibited by up-regulation of Hif3alpha. As cartilage degradation and hypertrophy are two hallmarks of osteoarthritis progression, it is not surprising that PRG4 has chondroprotective effects both in age-related and post-injury osteoarthritis (FIG. 4F).

Osteoarthritis Gene Therapy can be Enhanced by Combined Gene Transfer of PRG 4 and Il-1 Ra

The inventors sought to evaluate whether the beneficial effect of over-expressing PRG4 in osteoarthritis joints can be further improved by combining it with gene therapy mediated expression of Il-1Ra. Il-1Ra blocks the effects of Il-1beta, which is one of the key drivers of inflammation and cartilage catabolism in osteoarthritis. Based on the different pathways that PRG4 and Il-1 Ra exert their effects on, a combination of both might result in optimized inhibition of both cartilage breakdown and inflammation.

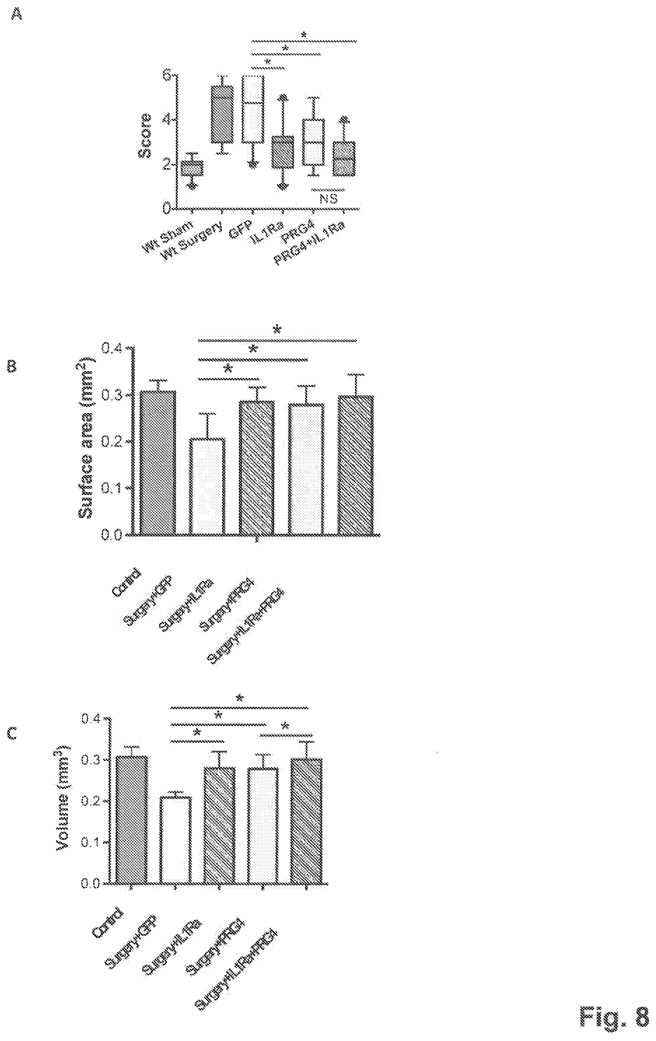

Mice had osteoarthritis induced and were injected with gene therapy vectors two weeks later. HDAd-PRG4. HDAd-Il-1Ra and the combination of both resulted in significantly lower osteoarthritis histology scores compared to the control vector HDAd-GFP and the no treatment group (FIG. 8A). Cartilage surface area was significantly higher in the HDAd-PRG4, HDAd-Il-1Ra and combination group compared with HDAd-GFP (FIG. 8B). Furthermore, cartilage volume was significantly higher in the HDAd-PRG4, HDAd-Il-1Ra and combination group compared with HDAd-GFP (FIG. 8C). The combination of HDAd-PRG and HDAd-Il-1Ra resulted in significantly higher cartilage volume compared with single vector treatment. The combination therapy resulted also in lower average osteoarthritis score and higher average cartilage surface area; however, the difference in these parameters did not reach statistical significance although a trend to significance was present.

### DISCUSSION

The invention shows by using both transgenic mice expressing Proteoglycan 4 (PRG4), and intra-articular, helper-dependent adenoviral virus (HDAd) gene transfer that PRG4 is protective against the development of both post-traumatic and age-related osteoarthritis, without significant adverse effects on cartilage development. Gene therapy treatment with HDAd-PRG4 was effective when injected before and after onset of osteoarthritis suggesting that the treatment is both preventive and therapeutic. The beneficial effect can be further improved by combining PRG4 with anti-inflammatory Il-1Ra gene therapy. The protective effects are demonstrated at molecular, histological and functional levels. The inventors further show that PRG4 over-expression inhibits transcriptional programs that promote cartilage catabolism and hypertrophy in part through the up-regulation of Hif3alpha. The concordant changes of PRG4 and HIF3alpha expression is also observed in gene expression profiling in human osteoarthritic patient samples.

Most genetics models reported to date show protection from osteoarthritis using histological endpoints at one month after surgical destabilization of the medical meniscus (DMM) to induce a mild, single condylar post-traumatic osteoarthritis, In addition, studies on osteoarthritis have been largely focused on loss of function mutations of genes in bone development such as Adamts5, Mmp13, Hif2alpha and Syndecan4 (F. Echtermeyer et al., Syndecan-4 regulates ADAMTS-5 activation and cartilage breakdown in osteoarthritis. Nature Medicine, 1 (Mar. 30, 2102); T. Saito et al., Transcriptional regulation of endochondral ossification by HIF-2α during skeletal growth and osteoarthritis development. Nature Medicine 16, 678 (Jun. 23, 2010); S, S. Glasson et al., Deletion of active ADAMTS5 prevents cartilage degradation in a murine model of osteoarthritis. Nature 434, 644 (Apr. 31, 2005); C. B. Little et al., Matrix metalloproteinase 13-deficient mice are resistant to osteoarthritic cartilage erosion but not chondrocyte hypertrophy or osteophyte development. Arthritis & Rheumatism 60, 3723 (December, 2009)). In contrast to these studies, the inventors report the gain of function genetic model with a secreted protein PRG4 that protects against osteoarthritis development at least 2 months after transection of cruciate ligaments, This model mimics a common injury in humans and leads to osteoarthritis in both condylar structures of the knee. The establishment of a gain of function model using an endogenously produced secreted protein may make for easier clinical translation as compared to previous approaches targeting inhibition of specific matrix enzymes and/or intracellular transcription factors. Moreover, the demonstration of a beneficial effect on age-related cartilage changes supports the further study of this approach beyond injury model.

The established mechanisms that protect animals from osteoarthritis development mostly depend on inhibition of cartilage catabolic enzymes. ADAMTS5 was the first target to be discovered via in vivo genetic experiments (S. S. Glasson et al., Deletion of active ADAMTS5 prevents cartilage degradation in a murine model of osteoarthritis. Nature 434, 644 (Apr. 31, 2005)). Loss of Syndecan 4, similarly, works through ADAMTS5 inhibition (F. Echtermeyer et al., Syndecan-4 regulates ADAMTS-5 activation and cartilage breakdown in osteoarthritis. Nature Medicine, 1 (Mar. 30, 2102)). Recently, the discovery of the protective effects of Hif2alpha loss of function in OA extends this approach as Hif2alpha transcriptionally regulates the expression of catabolic enzymes including several MMPs and ADAMTSs (T. Saito et al., Transcriptional regulation of endochondral ossification by HIF-2α during skeletal growth and osteoarthritis development. Nature Medicine 16, 678 (Jun. 23, 2010)). However, targeting anabolic pathways, including cell growth, differentiation and matrix synthesis, is equally important in osteoarthritis since chondrocyte proliferation, metaplasia and abnormal matrix synthesis have been long observed in osteoarthritis progression (K. P. Pritzker et al., Osteoarthritis cartilage histopathology: grading and staging. Osteoarthritis Cartilage 14, 13 (January, 2006)). An interaction between cartilage anabolic and catabolic pathways is required to maintain homeostasis and their imbalance leads to osteoarthritis progression. A therapy that can affect both programs would potentially be most effective.

Low-grade inflammation is commonly observed in osteoarthritic joints (Felson D T. 2006. Clinical practice. Osteoarthritis of the knee. N Engl J Med 354:841-848.). Besides maintaining and amplifying inflammation, the key inflammatory mediators in osteoarthritis such as Il-1beta also trigger the expression of cartilage degrading enzymes such as collagenases and aggrecanases (Daheshia, M., and YAO, J. Q. (2008). The Interleukin 1β Pathway in the Pathogenesis of Osteoarthritis. J Rheumatol 35, 2306.). Therefore, it seems important to inhibit both cartilage catabolism and joint inflammation in order to achieve efficient osteoarthritis treatment. Along these lines, the inventors show here that a gene therapy treatment combining helper-dependent adenoviral vectors expressing PRG4 and the anti-inflammatory Il-1Ra seems to further improve osteoarthritis treatment over gene therapy with PRG4.alone.

Materials and Methods

Generation of transgenic mice. FVB/N mice were purchased from Jackson Laboratories (Bar Harbor, ME). This strain is the common background strain for transgenic mouse lines. All studies were performed with approval from the Baylor College of Medicine Institutional Animal Care and Use Committee (IACUC). All mice were housed under pathogen-free conditions in less than five per cage. Mice had free access to feed and water. Transgenic mice were generated by pronuclear microinjection. Founders were outcrossed for at least 3 generations to eliminate multiple insertions. Different lines were tested at the beginning to rule out position effect. Genotyping primers were designed to detect the VVPRE element in the transgene cassette: F: TCTCTTTATGAGGAGTTGTGGCCC (SEQ ID NO: 40), R: CGACAACACCACGGAATTGTCAGT (SEQ ID NO: 41). To avoid the effects of potential post-menopausal bone loss, all the mice used in OA evaluation were males.

Cruciate ligament transection (CLT) surgery. CLT surgery and sham were performed as previously described in 8-week old male FVB/N mice and PRG4 transgenic mice (M. Ruan et al., Quantitative imaging of murine osteoarthritic cartilage by phase contrast micro-computed tomography. Arthritis Rheum, (2012). Investigators were blinded to the genotype of the mice when surgery was performed.

Histology and immunohistochemistry. Mice were euthanized and samples were fixed with 4% paraformaldehyde (Sigma-Aldrich) overnight in 4° C. on a shaker. Samples from mice older than 4 days were decalcified in 14% EDTA for 5 days in 4° C. on a shaker. Samples from mice younger than 3 days were not decalcified. Paraffin embedding was performed as previously described. Samples were sectioned at 6 μm. Samples were stained with safranin O and fast green using standard protocols. Samples were scored by two independent pathologists masked to the procedure and genotypes. Immunohistochemistry were performed using primary antibody: anti-PRG4 (Abcam, ab 28484), anti-MMP13 (Millipore, MAB 13424), anti-ColX (generous gift from Dr. Greg Lunstrum, Shriners Hospital for Children, Portland, OR), and secondary antibody: one-dropper-bottle HRP polymer conjugates (Invitrogen). BrdU staining was performed using anti-BrdU Alexa Fluor 594 (A21304, Invitrogen). Histomark trueblue (KPL) was used as developing reagent. TUNEL staining was performed using ApopTag Plus Peroxidase In situ Apoptosis Detection (Millipore Kit S7101) following manufacturer's protocol. All staining in the same experiment were done at the same time. Observer who quantified of BrdU and TUNEL staining was blinded to the genotype of the mice.

Beta-galactosidase staining. Staining was performed on samples embedded in optimal cutting temperature compound after fixation and decalcification. Samples were sectioned at 6 μm and stained with X-gal (X428IC Gold biotechnology) overnight and nuclear fast red (N3020 Sigma) as counter stain.

Rotarod analysis. Mice were placed onto an accelerating rotarod (UGO Basile, Varese, Italy). The duration to first failure to stay atop the rod was marked as first ride-around time. To rule out differences in learning skills between the two groups of mice, each group was assessed over three trials per day for 2 consecutive days (trials 1 to 6) before surgery. Mice were then randomly assigned into different groups. Another 6 trials were performed using the same conditions at the different time points after the surgery. Mice were given a minutes inter-trial rest interval. Each trial had a maximum time of 5 minutes. Observer was blinded to the genotype and the procedure of the mice.

Hotplate analysis. Mice were placed on the hotplate at 55° C. (Columbus Instruments, Columbus, OH). The latency period for hind limb response (e.g. shaking, jumping, or licking) was recorded as response time before at different time points after surgery. Observer was blinded to the genotype and the procedure of the mice.

Phase contrast μCT scanning. Samples were prepared as previously described and scanned by Xradia μXCT at source voltage=40 kV, source power=8W, detector distance from sample=75 mm, source distance from sample=100 mm, image number taken=500, and exposure time for each image=30 (M. Ruan et al., Quantitative imaging of murine osteoarthritic cartilage by phase contrast micro-computed tomography. Arthritis Rheum, (2012)). The resolution of the scanning is 4 μm. After scanning, a random number was assigned to each sample to ensure blinded assessment during image processing.

Reconstruction and analysis of μCT data. Reconstruction of the data was performed using Xradia software and was transformed into dicom files. Reconstruction involves correction for beam hardening (constant=0.3), and correcting for center shift effects caused by difference between the center of sample rotation and the center of the detector. Samples were analyzed using TriBON software (RATOC, Tokyo, Japan). Observers were blinded to the procedure and sample number (M. Ruan et al., Quantitative imaging of murine osteoarthritic cartilage by phase contrast micro-computed tomography. Arthritis Rheum, (2012)).

Intra-articular Injection. Mice were anesthetized using 3% isoflurane. Joint area was shaved. HDAds were diluted in sterile PBS in 5 μl and injected by 25 μl CASTIGHT syringes (1702 Hamilton Company) and 33 gauge needles (7803-05 Hamilton Company).

Luciferase assay. Mice were injected with 2 mg D-luciferin (L9504 SIGMA) diluted in 100 μl PBS per mouse (25 grams) intraperitoneally. Mice were anesthetized using 3% isoflurane. Images were taken by Xenogen IVIS optical in vivo imaging system. Quantification was performed by living Imaging 4.2 using default settings. Image was collected for 10 minutes after the injection and normalized to control mice without luciferase injection.

Laser capture microdissection and RNA purification. Hind limbs of P1 littermates were collected and snap-frozen in liquid nitrogen. Then, samples were embedded in optimal cutting temperature compound. Frozen sections of 10 μm were generated on polyethylene napthalate (PEN)-membrane slides. Superficial layer chondrocytes were captured using HS Capsure LCM caps by Applied Biosciences Acturus Systems. RNA was then purified by Picopure RNA isolation kit.

Mouse Microarray and analysis. Microarrays were performed using Mouse WG-6 v2.0 Expression BeadChip (Illumina). Data was processed using the lumi package within the R statistical package. Variance-stabilizing trans-formation (VST) was performed, followed by quantile normalization of the resulting expression values. Differential expression was calculated using the limma package within R. Heat map was generated using normalized fold change. The resulting lists were then annotated and reviewed for candidates.

Human gene expression analysis. GEO archives GSE10575 titled “Migratory chondrogenic progenitor cells from repair tissue during the later stages of human osteoarthritis” (PMID: 19341622) and GSE16464, titled “Chondrogenic differentiation potential of osteoarthritis chondrocytes and their use in autologous chondrocyte transplantation” (PMID: 19723327) were both downloaded and analyzed using the web-based GEO2R, using the default settings, available through the GEO site. In archive GSE10575, three arrays of chondrogenic progenitor cells from osteoarthritis males were compared to two control arrays of the same cell type. Female samples were excluded because control samples are males (shown by the level of Xist expression). In archive GSE 16464, 3D-cultured chondrocytes from normal donors and 3D-cultured chondrocytes from OA donors were compared. Both archives used the Affymetrix Human Genome U133 Plus 2.0 Array platform.

Cell culture, transfection and infection. C3H10T1/2 cells were maintained in DMEM with 10% FBS; TC71 cells were maintained in RPMI 1640 with 10% FBS; ATDC5 cells were maintained in DMEM/F-12 1:1 mixture supplemented with 10% FBS. Cells were plated the day before transfection/infection so that it reached 70% confluency the next day. Lipofectamine 2000 was used as transfection reagent following protocols provided by manufacturer. Dharmacon on-target siRNA was used in the knockdown assay. HDAds were generated as previously described (M. Suzuki et al., Large-scale production of high-quality helper-dependent adenoviral vectors using adherent cells in all factories. Human gene therapy 21,120 (February, 2010)). HDAd-PRG4 carries the murine PRG4 gene controlled by the constitutive EF1 promoter. HDAd-Il-1Ra carries the murine Il-1Ra gene controlled by an inflammation-inducible NF-κB promoter. To infect cells. HDAds were diluted at 5000 vp/cell and added in serum free media with minimal volume covering cells after aspiration. Two hours later, media containing virus was aspirated and culturing media was added back. For hypoxia experiments, cells were transferred to hypoxia chamber with 1% oxygen.

RNA purification and quantitative PCR. Cells were lysed with Trizol reagents (Invitrogen) and RNA was purified following manufacturer's protocol. To eliminate DNA contamination, samples were treated with RNase-free recombinant DNaseI (Roche). Reverse-transcript PCR was conducted by superscript III first strand (18080-051Invitrogen) following manufacturer's protocol. Taqman Universal PCR mastermix (Applied biosciences) and PerfeCTa SYBR Green SuperMix (Quanta BioSciences) were used in quantitative PCR. Primers used in quantitative PCR are listed as follows: mouse PRG4: F:ACTTCAGCTAAAGAGACACGGAGT (SEQ ID NO: 16), R: GTTCAGGTGGTTCCTTGGTTGTAGTAA (SEQ ID NO: 17); Sox9: F: AAGCCACACGTCAAGCGACC (SEQ ID NO: 18), R: GTGCTGCTGATGCCGTAACT (SEQ ID NO: 19); Col2a1; F: GCTCATCCAGGGCTCCAATGATGTAG (SEQ ID NO: 20), R: CGGGAGGTCTTCTGTGATCGGTA (SEQ ID NO: 21); Gapdh: F: GCAAGAGAGGCCCTATCCCAA (SEQ ID NO: 22); R: CTCCCTAGGCCCCTCCTGTTATT (SEQ ID NO: 23); Vegf: F: TGGACTTGTGTTGGGAGGAGGATG (SEQ ID NO: 24), R: GCCTCTTCTTCCACCACCGTGTC (SEQ ID NO: 25); Mmp13: F: GCAATCTTTCTTTGGCTTAGAGGT (SEQ ID NO: 26), R: GGTGTTTTGGGATGCTTAGGGT (SEQ ID NO: 27); Col10al: F: AAAGCTTACCCAGCAGTAGG (SEQ ID NO: 28), R: ACGTACTCAGAGGAGTAGAG (SEQ ID NO: 29); GAPDH: F: ATACCAGGAAATGAGCTTGACAAA (SEQ ID NO: 30), R: TGAAGGTCGGAGTCAACGGA (SEQ ID NO: 31); VEGF: F: GATCGGTGACAGTCACTAGCTTATCT (SEQ ID NO: 32), R: TACACACAAATACAAGTTGCCA (SEQ 1D NO: 33); MMP13 F: TGCCCTTCTTCACACAGACACTAACGAAA (SEQ ID NO: 34), R: GGCCACATCTACTATTCTTACCACTGCTC (SEQ ID NO: 35); COL10A1: F: GCCCACTACCCAAGACCAAGAC (SEQ ID NO: 36); R: GACCCCTCTCACCTGGACGAC (SEQ ID NO: 37); HIF3A: F: GGCTGTTCCGCCTACGAGTA (SEQ ID NO: 38); R: AGCAAGGTGGATGCTCTTG (SEQ ID NO: 39); PRG4: Hs00981633_m1 (applied biosciences); mouse Hif3a: Mm00469375_m1 (applied biosciences).

Statistics. Statistical significance comparing two groups with parametric data was assessed by Student's t test, Statistical analysis comparing multiple groups with parametric data was performed by one-way ANOVA followed by Tukey's post-hoc. Statistical analysis comparing different genotype with different procedure was performed by two-way ANOVA followed by Tukey's post-hoc, Normality was tested by Shapiro-Wilk Normality test. Histological grades were compared by Wilcox rank test. All analyses were performed by SPSS software or Sigma Plot. A P value of <0.05 was considered statistically significant.

Dataset 1: List of genes showing more than 1.5 fold change in the microarray analysis.

| | WTP1_1 | WTP1_2 | WIP1_3 | PG4P1_1 | PG4P1_2 | PG4P1_3 |
|---|---|---|---|---|---|---|
| 4930546H06Rik_ILMN_2717117 | 1.256363 | 1.360731 | 1.425929 | −1.48447 | −1.59768 | −1.52108 |
| Sesn_ILMN_2654074 | −1.25604 | −1.26072 | −1.28391 | 1.234927 | 1.170478 | 1.226371 |
| Ypel5_ILMN_1251071 | 1.224517 | 1.294585 | 1.311664 | −1.44517 | −1.32632 | −1.38253 |
| Bglap-rs1_ILMN_1233122 | 1.301868 | 2.023561 | 1.628096 | −3.0338 | −3.05449 | −2.56761 |
| AK038070_ILMN_2466021 | 1.257255 | 1.181603 | 1.204158 | −1.30391 | −1.20282 | −1.31808 |
| AK011460_ILMN_2452717 | −1.52912 | −1.85077 | −1.48354 | 1.474372 | 1.352955 | 1.304319 |
| Bglap1_ILMN_2610166 | 1.404137 | 2.037433 | 1.6235 | −4.09687 | −3.29889 | −2.57926 |
| Med18_ILMN_1214050 | −1.48231 | −1.26813 | −1.3871 | 1.212726 | 1.267995 | 1.335163 |
| Bglap1_ILMN_3101908 | 1.459117 | 2.042785 | 1.496434 | −3.82431 | −3.03438 | −2.43533 |
| Ppp1r3c_ILMN_2667091 | −1.65083 | −1.56199 | −1.48727 | 1.4376 | 1.167941 | 1.476123 |
| Bglap-rs1_ILMN_1220829 | 1.261849 | 2.094587 | 1.691356 | −3.64532 | −3.48459 | −2.55816 |
| Zfp46_ILMN_1215740 | −1.35114 | −1.26299 | −1.37143 | 1.277394 | 1.136789 | 1.32476 |
| Hist1h1c_ILMN_2855315 | 1.196666 | 1.423895 | 1.348674 | −1.49333 | −1.34167 | −1.62396 |
| Zfand2a_ILMN_1230489 | 1.273059 | 1.140929 | 1.34758 | −1.36636 | −1.26743 | −1.39361 |
| Ddx21_ILMN_2546724 | 1.305544 | 1.217222 | 1.179125 | −1.34811 | −1.20085 | −1.382 |
| Hist1h1c_ILMN_2774537 | 1.170861 | 1.384453 | 1.428754 | −1.41392 | −1.42079 | −1.65332 |
| Ddx54_ILMN_2689678 | 1.130087 | 1.286618 | 1.299302 | −1.39565 | −1.2461 | −1.30723 |
| Rps15a_ILMN_2717621 | 1.315098 | 1.139016 | 1.449143 | −1.36561 | −1.52841 | −1.40807 |
| Bglap-rs1_ILMN_2944508 | 1.303982 | 2.185122 | 1.520279 | −3.70241 | −3.45533 | −2.31957 |
| Slc7a5_ILMN_2711948 | 1.201827 | 1.326767 | 1.430059 | −1.67471 | −1.48398 | −1.29808 |
| Csnk1d_ILMN_2739965 | 1.11045 | 1.255629 | 1.292397 | −1.37356 | −1.22945 | −1.24982 |
| Dyrk1b_ILMN_3053158 | 1.46521 | −1.31006 | −1.19201 | 1.182241 | 1.294858 | 1.238163 |
| Hist2h3b_ILMN_2934120 | 1.254913 | 1.119073 | 1.304197 | −1.38431 | −1.299 | −1.20538 |
| Nmd3_ILMN_1228859 | 1.262366 | 1.122484 | 1.224622 | −1.20301 | −1.36721 | −1.20793 |
| 1190005F20Rik_ILMN_2697918 | −1.21616 | −1.40512 | −1.44745 | 1.36511 | 1.269442 | 1.140632 |
| Lrrc59_ILMN_1252817 | 1.334493 | 1.222121 | 1.168151 | −1.47501 | −1.29518 | −1.21186 |
| Dusp8_ILMN_1228031 | 1.288724 | 1.069707 | 1.275318 | −1.27037 | −1.24915 | −1.28446 |
| AK053260_ILMN_1228804 | −1.53513 | −1.52115 | −1.78747 | 1.594074 | 1.424237 | 1.113431 |
| Eif1a_ILMN_2698107 | 1.399002 | 1.348068 | 1.07882 | −1.44405 | −1.34553 | −1.35426 |
| AK021349_ILMN_1258961 | 1.382804 | 1.270095 | 1.173873 | −1.34656 | −1.59361 | −1.2452 |
| Csnk1d_ILMN_1231035 | 1.104983 | 1.305878 | 1.354114 | −1.47277 | −1.23751 | −1.33697 |
| Cirbp_ILMN_2761594 | 1.08657 | 1.233789 | 1.298241 | −1.23605 | −1.35285 | −1.20021 |
| Plekhf2_ILMN_2798694 | 1.124677 | 1.171659 | 1.32521 | −1.2892 | −1.34384 | −1.16463 |
| Nfatc1_ILMN_1216522 | 1.286293 | 1.080945 | 1.308693 | −1.21468 | −1.39646 | −1.27436 |
| Clk1_ILMN_2428301 | −1.36643 | −1.38464 | −1.9415 | 1.450046 | 1.301662 | 1.279182 |
| Trove2_ILMN_1252725 | 1.195356 | 1.139589 | 1.272322 | −1.22692 | −1.41043 | −1.15117 |
| EG639396_ILMN_2877059 | −1.19236 | −1.20876 | −1.39289 | 1.317211 | 1.11903 | 1.179859 |
| Heatr1_ILMN_1214036 | 1.317403 | 1.264432 | 1.223294 | −1.64477 | −1.32574 | −1.20107 |
| BC027809_ILMN_1252263 | 1.357622 | 1.055984 | 1.444709 | −1.34453 | −1.47672 | −1.38744 |
| Ccdc130_ILMN_2756733 | −1.17101 | −1.26787 | −1.41093 | 1.342647 | 1.169009 | 1.136904 |
| Tmcc1_ILMN_1249710 | −1.26174 | −1.39677 | −1.70812 | 1.477478 | 1.22952 | 1.199067 |
| Arrdc4_ILMN_2648967 | 1.045261 | 1.574252 | 1.593893 | −1.83439 | −1.7299 | −1.50742 |
| AK044963_ILMN_1247942 | 1.321698 | 1.257721 | 1.270333 | −1.18779 | −1.38985 | −1.69824 |
| Arf2_ILMN_1214810 | 1.546019 | 1.353128 | 1.087012 | −1.4972 | −1.66445 | −1.34205 |
| 2610101N10Rik_ILMN_1252490 | −1.14039 | −1.40709 | −1.51568 | 1.290925 | 1.162057 | 1.299667 |
| Lrrc47_ILMN_2628551 | 1.117726 | 1.123233 | 1.383986 | −1.31904 | −1.23011 | −1.2438 |
| Srm_ILMN_2809611 | 1.485302 | 1.196046 | 1.150938 | −1.54798 | −1.39998 | −1.23852 |
| Gprc5a_ILMN_2854943 | 1.320606 | 1.042922 | 1.353049 | −1.29647 | −1.25915 | −1.39294 |
| Ddx11_ILMN_2700550 | −1.20131 | −1.24547 | −1.33501 | 1.353463 | 1.204601 | 1.057541 |
| Deb1_ILMN_2652971 | 1.548657 | 1.086404 | 1.190474 | −1.36664 | 1.38753 | −1.38497 |
| Mif_ILMN_2867835 | 1.125532 | 1.187658 | 1.327057 | −1.3999 | −1.32759 | −1.12086 |
| Hoxd3_ILMN_1219807 | −1.19061 | −1.30954 | −1.55302 | 1.334377 | 1.316337 | 1.101846 |
| Cdc7_ILMN_1238374 | −1.31242 | −1.26666 | −1.86596 | 1.253473 | 1.281007 | 1.378172 |
| Zfp187_ILMN_3067831 | −1.18727 | −1.40885 | −1.54414 | 1.457655 | 1.221198 | 1.121467 |
| 3300001P08Rik_ILMN_2727004 | −1.16879 | −1.39116 | −1.32235 | 1.224787 | 1.064016 | 1.330551 |
| Hoxc6_ILMN_1217328 | −1.33051 | −1.47422 | −1.40075 | 1.339994 | 1.496705 | 1.019485 |
| Rbm5_ILMN_2942492 | −1.29365 | −1.62199 | −1.68464 | 1.66198 | 1.236204 | 1.11868 |
| AK012053_ILMN_2469320 | −1.10834 | −1.3099 | −1.40348 | 1.32378 | 1.110387 | 1.187654 |
| Tmem128_ILMN_1248235 | 1.125965 | 1.138522 | 1.441919 | −1.45467 | −1.29997 | −1.19488 |
| Rbm5_ILMN_2942499 | −1.45228 | −1.59992 | −1.84238 | 1.607189 | 1.534819 | 1.001613 |
| E130016E03Rik_ILMN_3161959 | −1.2783 | −1.50572 | −1.71101 | 1.568022 | 1.337492 | 1.063614 |
| Tnfrsf12a_ILMN_2424299 | 1.680884 | 1.277593 | 1.075987 | −1.78329 | −1.46531 | −1.38442 |
| Prdm2_ILMN_1250454 | 1.173017 | 1.20632 | 1.256779 | −1.14469 | −1.19644 | −1.52795 |
| Srm_ILMN_1225880 | 1.332372 | 1.120064 | 1.166016 | −1.4506 | −1.22663 | −1.14034 |
| Cpt2_ILMN_2775123 | −1.20532 | −1.33083 | −1.38724 | 1.104396 | 1.131549 | 1.462135 |
| Twf1_ILMN_1244219 | 1.25757 | 1.126966 | 1.235596 | −1.49415 | 1.20089 | −1.13911 |
| AK076052_ILMN_2429108 | 1.076837 | 1.140505 | 1.40668 | −1.19618 | −1.25719 | −1.34308 |
| Farsa_ILMN_1257639 | 1.213878 | 1.216622 | 1.352934 | −1.12429 | 1.38604 | 1.65116 |
| Zfp84_ILMN_2506757 | −1.16964 | −1.18995 | −1.49011 | 1.301893 | 1.104954 | 1.226727 |
| Rnase4_ILMN_1235657 | −1.46403 | −1.24206 | −1.12261 | 1.323919 | 1.163284 | 1.133851 |
| Pim3_ILMN_2717667 | 1.123427 | 1.144501 | 1.541166 | −1.42121 | −1.21509 | −1.50536 |
| Rab5b_ILMN_1237467 | −1.54934 | −1.50901 | −1.79875 | 1.824154 | 1.27382 | 1.037959 |
| Tk1_ILMN_2605890 | −1.14797 | −1.19726 | −1.52316 | 1.200172 | 1.303368 | 1.13359 |
| Nrp1_ILMN_1247094 | −1.34674 | −1.14618 | −1.60363 | 1.244615 | 1.403199 | 1.113605 |
| 6030458C11Rik_ILMN_1234196 | −1.5397 | −1.45778 | −2.22228 | 1.702852 | 1.461039 | 1.050668 |
| Pold1_ILMN_2655577 | −1.24471 | −1.24541 | −1.4781 | 1.425738 | 1.253099 | 1.038272 |
| Prss35_ILMN_2609897 | 1.480951 | −1.03832 | 1.608742 | −1.44299 | −1.64698 | −1.54549 |
| Tppp3_ILMN_2655929 | −1.08048 | −1.39071 | −1.3892 | 1.218015 | 1.318692 | 1.098879 |
| Clec2d_ILMN_2603647 | −1.73457 | −1.2971 | −2.52661 | 1.280941 | 1.696474 | 1.279334 |
| Hif3a_ILMN_2649671 | −1.78312 | −1.6431 | −1.32223 | 1.73224 | 1.313779 | 1.028263 |
| Pnrc2_ILMN_2861331 | −1.12969 | −1.18773 | −1.58246 | 1.161941 | 1.19852 | 1.280471 |
| Myd116_ILMN_2722938 | 1.38863 | 1.149001 | 1.09759 | −1.13863 | −1.23126 | −1.4829 |
| Eln_ILMN_2697304 | 1.09603 | 1.310437 | 1.235098 | −1.20273 | −1.1346 | −1.54888 |
| Fbxo31_ILMN_2452855 | −1.16259 | −1.1719 | −1.62377 | 1.23252 | 1.296053 | 1.142115 |
| Zmym2_ILMN_2781493 | −1.1322 | −1.205 | −1.65697 | 1.259404 | 1.17403 | 1.249941 |
| Rad541_ILMN_2741985 | −1.14435 | −1.14233 | −1.57512 | 1.210268 | 1.2324 | 1.17319 |
| Nedd9_ILMN_2654186 | 1.300471 | 1.369214 | 1.006802 | −1.27054 | −1.16693 | −1.47168 |
| Gadd45g_ILMN_2744890 | 1.159873 | 1.093824 | 1.401321 | −1.50495 | −1.10425 | −1.29047 |
| Syncrip_ILMN_1258420 | 1.212463 | 1.238146 | 1.1686 | −1.42916 | −1.42482 | −1.02121 |
| ler3_ILMN_1216764 | 1.881239 | 1.019378 | 1.168521 | −1.48139 | −1.66306 | −1.52784 |
| Galt_ILMN_2677567 | −1.21625 | −1.36275 | −1.40341 | 1.540977 | 1.172451 | 1.018011 |
| Map3k1_ILMN_2614380 | −1.22523 | −1.25855 | −1.72004 | 1.286516 | 1.479816 | 1.041544 |
| Rrp12_ILMN_2728118 | 1.330091 | 1.265982 | 1.182798 | −1.73253 | −1.45231 | −1.0467 |
| Dbp_ILMN_2616226 | −1.3989 | −1.2558 | −2.17137 | 1.654516 | 1.166472 | 1.207322 |
| Sfrs7_ILMN_2552490 | −1.09319 | −1.37266 | −1.50612 | 1.421972 | 1.225556 | 1.045249 |
| Tmem100_ILMN_1224014 | −1.53254 | −1.20266 | −1.67189 | 1.499528 | 1.458731 | −1.04208 |
| Rpap1_ILMN_1238065 | 1.22594 | −1.19869 | −1.41188 | 1.480394 | 1.120412 | 1.040978 |
| AK017419_ILMN_2416218 | 1.258403 | −1.04994 | 1.597254 | −1.40368 | −1.39738 | −1.30912 |
| Trpc2_ILMN_1220948 | −1.1478 | −1.20737 | −1.48702 | 1.435121 | 1.116418 | 1.076493 |
| Il11ra1_ILMN_1229957 | −1.17181 | −1.34116 | −1.36512 | 1.51528 | 1.033071 | 1.120113 |
| AK007736_ILMN_1258587 | −1.30198 | −1.1931 | −1.5084 | 1.02934 | 1.151676 | 1.549816 |
| Atpbd4_ILMN_1230688 | 1.212165 | 1.044319 | 1.518026 | −1.1629 | −1.29912 | −1.67838 |
| Tuba1c_ILMN_2476139 | 1.185176 | 1.22582 | 1.255489 | −1.65104 | −1.03071 | −1.31991 |
| Abhdl4b_ILMN_3007862 | −1.17428 | −1.31793 | −1.82363 | 1.555108 | 1.207257 | 1.078925 |
| Ifi2712a_ILMN_2762944 | 1.023728 | 1.619691 | 1.164394 | −1.58706 | −1.19828 | −1.37445 |
| MII5_ILMN_1217776 | −1.08848 | −1.35305 | −1.70073 | 1.363244 | 1.345082 | 1.045908 |
| AK029312_ILMN_1246692 | 1.1321 | −1.22047 | −1.47253 | 1.301738 | −1.00965 | 1.326047 |
| Cars2_ILMN_1218543 | −1.15407 | −1.26682 | −1.75735 | 1.471686 | 1.249823 | 1.053571 |
| Raver2_ILMN_1213278 | −1.20699 | −1.23934 | −1.94009 | 1.342784 | 1.066234 | 1.440154 |
| Mid1_ILMN_3159435 | 1.127098 | 1.038972 | 1.460931 | −1.13274 | −1.25072 | −1.44792 |
| AK078053_ILMN_1222351 | −1.10161 | −1.27506 | −1.55656 | 1.45516 | 1.108898 | 1.101466 |
| AK028672_ILMN_1246030 | 1.223792 | 1.161685 | 1.206628 | −1.02194 | −1.2642 | −1.56654 |
| AK043421_ILMN_2575994 | −1.26016 | −1.2699 | −2.19263 | 1.481949 | 1.406903 | 1.07406 |
| AK085118_ILMN_2505392 | −1.11638 | −1.20699 | −1.48486 | 1.360363 | 1.232508 | 1.009408 |
| Lpp_ILMN_2463260 | 1.144334 | 1.172656 | 1.289494 | 1.004724 | −1.44657 | −1.43369 |
| NR_001461_ILMN_2445958 | −1.33534 | −1.58378 | −1.69795 | 1.861753 | 1.221821 | −1.05574 |
| Zfp52_ILMN_2838139 | −1.25077 | −1.21913 | −1.67899 | 1.544861 | 1.244823 | −1.00507 |
| AK004187_ILMN_2513451 | −1.30128 | −1.18771 | −1.30172 | 1.256082 | 1.421114 | −1.05914 |
| Gp1bb_ILMN_2653205 | 1.310991 | −1.01749 | 1.499583 | −1.20277 | −1.26174 | −1.71629 |
| BC037034_ILMN_1257019 | −1.07956 | −1.19392 | −1.58424 | 1.197539 | 1.32752 | 1.079839 |
| AK086317_ILMN_2580737 | 1.974206 | 1.266016 | 1.059287 | −1.17028 | −2.25587 | −2.48319 |
| NR_002848_ILMN_2966602 | −1.34056 | −1.63363 | −1.01451 | 1.281704 | 1.267341 | 1.107163 |
| Asns_ILMN_2643513 | 1.12627 | 1.082326 | 1.473384 | −1.63603 | −1.22191 | −1.12562 |
| AK005089_ILMN_2451115 | 1.054101 | 1.319016 | 1.421071 | −1.13881 | −1.24881 | −1.89777 |
| 6430706D22Rik_ILMN_3011719 | −1.33993 | −1.32228 | −1.58013 | 1.622833 | 1.345146 | −1.11534 |
| 5330401P04Rik_ILMN_2520011 | −1.1508 | −1.17966 | −1.63107 | 1.427003 | 1.221601 | 1.021642 |
| Flcn_ILMN_1213483 | −1.17513 | −1.1274 | −1.55513 | 1.410583 | 1.179933 | 1.028483 |
| Per2_ILMN_2987862 | −1.21694 | −1.06498 | −1.65428 | 1.267989 | 1.067504 | 1.299293 |
| Ppm1m_ILMN_1224437 | −1.16175 | −1.17068 | −1.91121 | 1.437044 | 1.159707 | 1.165048 |
| AK084113_ILMN_2451389 | −1.16976 | −1.13767 | −1.70942 | 1.283512 | 1.371085 | 1.026543 |
| AK007605_ILMN_1239776 | 1.119895 | 1.118125 | 1.47604 | −1.14506 | −1.18395 | −1.76057 |
| Depdc6_ILMN_3163001 | −1.13115 | −1.20253 | −1.78263 | 1.08135 | 1.165515 | 1.47653 |
| Mterf_ILMN_2624809 | −1.0454 | −1.20426 | −1.5987 | 1.344071 | 1.147785 | 1.095675 |
| AK014695_ILMN_2748880 | −1.23553 | −1.24466 | −2.42903 | 1.564719 | 1.31189 | 1.098905 |
| Vat1l_ILMN_1226356 | −1.17602 | −1.20391 | −1.46333 | 1.144397 | −1.02032 | 1.511196 |
| Ankzf1_ILMN_2703321 | −1.20097 | −1.19701 | −1.82856 | 1.447617 | 1.36439 | −1.02771 |
| Tha1_ILMN_2594768 | −1.09096 | −1.25311 | −1.78214 | 1.402023 | 1.303303 | 1.018911 |
| Plekhf1_ILMN_2993334 | −1.08624 | −1.16026 | −1.78844 | 1.297336 | 1.242747 | 1.118284 |
| Gprasp1_ILMN_3142384 | −1.15055 | −1.14972 | −1.66918 | 1.014435 | 1.224737 | 1.422807 |
| Pdgfra_ILMN_1235932 | −1.28478 | −1.3484 | −1.50982 | 1.62719 | 1.311287 | −1.13736 |
| Mcm10_ILMN_2970532 | −1.0688 | −1.14651 | −1.70048 | 1.161108 | 1.296402 | 1.146574 |
| Fbp2_ILMN_2634905 | −1.67939 | −1.76924 | 2.20592 | −1.36206 | 1.508826 | 2.142995 |
| 111007M04Rik_ILMN_N2734060 | 1.125411 | 1.216839 | 1.439906 | −1.77901 | −1.60341 | 1.032062 |
| Suv420h2_ILMN_1260420 | −1.06155 | −1.32955 | −2.05337 | 1.396444 | 1.349127 | 1.073276 |
| Adat2_ILMN_2705097 | 1.042499 | 1.080097 | 1.530343 | −1.07116 | −1.36781 | −1.46542 |
| 1200016B10Rik_ILMN_1236716 | −1.08228 | −1.17359 | 1.66229 | 1.410154 | 1.112488 | 1.099721 |
| AK021262_ILMN_2546861 | −2.55735 | −2.96551 | −2.04609 | 2.589175 | 1.589228 | −1.65394 |
| NR_002848_ILMN_2438819 | −1.04157 | −1.19208 | −1.62472 | 1.102605 | 1.130335 | 1.352612 |
| Iqcb1_ILMN_2635348 | −1.25858 | −1.51247 | −1.07587 | 1.479027 | 1.112991 | 1.022781 |
| Sirpa_ILMN_2722996 | −1.55085 | 1.020695 | −1.42422 | 1.095271 | 1.397855 | 1.139231 |
| Kif1b_ILMN_2587761 | −1.10748 | −1.26856 | −1.71852 | −1.03298 | 1.275994 | 1.482789 |
| Glpbp2_ILMN_2600113 | −1.03362 | −1.24214 | −1.75238 | 1.314034 | 1.280253 | 1.062528 |
| Akap81_ILMN_1242769 | −1.70634 | −1.79935 | −1.46855 | 1.863393 | 1.552434 | −1.31333 |
| AK029270_ILMN_1246021 | −1.36249 | −1.13168 | −2.29819 | 1.675332 | 1.15085 | 1.121103 |
| Timm8a1_ILMN_2896552 | 1.183976 | 1.065901 | 1.33591 | 1.5964 | −1.26281 | −1.0041 |
| Cpt2_ILMN_2775122 | −1.31568 | −1.57523 | −1.10064 | −1.0034 | 1.133167 | 1.566763 |
| Mif_ILMN_1260512 | 1.004975 | 1.323347 | 1.316609 | −1.560996 | −1.34456 | −1.0099 |
| Cenpl_ILMN_2676726 | −1.07561 | −1.16457 | −1.74209 | 1.300334 | 1.282306 | 1.05495 |
| Ccdc86_ILMN_2730003 | 1.079631 | 1.174875 | 1.493109 | −1.81679 | −1.39094 | −1.01727 |
| Cited2_ILMN_2477221 | −1;25941 | −1.07229 | −1.54732 | −1.01603 | 1.188502 | 1.45439 |
| Tle6_ILMN_2900617 | −1.25696 | −1.29766 | −2.43442 | 1.763195 | 1.23855 | 1.021292 |
| Mrm1_ILMN_2649654 | −1.12291 | −1.18885 | −1.88148 | 1.496945 | 1.102948 | 1.136915 |
| Tsc22d3_ILMN_3150811 | −1.46991 | −1.23229 | −1.52848 | 1.715752 | 1.257691 | −1.13571 |
| Wnk1_ILMN_1234955 | −1.1436 | −1.18321 | −1.62288 | 1.476736 | 1.220566 | −1.03422 |
| Gstt3_ILMN_2665715 | −1.15761 | −1.16266 | −1.67614 | 1.531124 | 1.053215 | 1.095113 |
| Ppm1k_ILMN_2923615 | −1.08558 | −1.15334 | −1.64931 | 1.404428 | 1.134997 | 1.066046 |
| Clk4_ILMN_2851710 | −1.15318 | −1.33127 | −1.79737 | 1.595963 | 1.307868 | −1.08522 |
| Il11ra2_ILMN_2619594 | −1.04918 | −1.28561 | −1.57792 | 1.475052 | 1.023734 | 1.136498 |
| Cars2_ILMN_2670601 | −1.09189 | −1.14083 | −1.62876 | 1.389033 | 1.161988 | 1.042614 |
| Pbx1_ILMN_2559669 | 1.2673 | −1.25299 | −2.278 | 1.703366 | 1.314599 | −1.04615 |
| Acot11_ILMN_1227579 | −1.16373 | −1.10948 | −1.73831 | 1.422989 | 1.008933 | 1.232184 |
| Neat1_ILMN_2493030 | −1.1433 | −1.74898 | −4.34058 | 1.651763 | 1.528512 | 1.142921 |
| AK020467_ILMN_2506727 | −1.09908 | −1.81523 | −1.30434 | 1.576618 | 1.226821 | −1.03184 |
| Calb2_ILMN_2827729 | −1.13166 | −1.24465 | −1.58857 | 1.188467 | 1.548766 | −1.05688 |
| Rassf4_ILMN_2956092 | −1.19773 | −1.02283 | −1.71608 | 1.100194 | 1.347195 | 1.157291 |
| Tia1_ILMN_1215055 | −1.07031 | −1.11087 | −1.74002 | 1.144017 | 1.141023 | 1.305753 |
| Csnk2a1_ILMN_1218670 | −1.22304 | −1.30599 | −2.00138 | 1.579869 | 1.464052 | −1.14536 |
| INV_ILMN_1257729 | −1.19494 | −1.45406 | −1.54665 | 1.780692 | 1.124428 | −1.08257 |
| Fam109a_ILMN_2668178 | −1.1457 | −1.10631 | −2.07282 | 1.126329 | 1.371624 | 1.242875 |
| Clspn_ILMN_2858359 | −1.09104 | −1.12459 | −2.01152 | 1.193798 | 1.211273 | 1.292024 |
| AK051059_ILMN_2419748 | −1.0608 | −1.16991 | −1.93243 | 1.379757 | 1.164043 | 1.141268 |
| Unc5c_ILMN_2461668 | −1.01081 | 1.174419 | 1.441474 | −1.35249 | −1.00552 | −1.51306 |
| AK078921_ILMN_2462678 | −1.11083 | −1.23212 | −1.74051 | 1.561371 | 1.188618 | −1.03775 |
| Sgk_ILMN_1213954 | 1.274151 | 1.55087 | −1.15069 | −1.29135 | −1.183 | −1.4572 |
| Disp1_ILMN_2772288 | −1.08894 | −1.41679 | −1.49469 | 1.632533 | 1.126658 | −1.05526 |
| Gadd45g_ILMN_2903945 | 1.451953 | 1.318414 | 1.04743 | −2.13919 | 1.014627 | −1.42835 |
| LOC100040259_ILMN_1244853 | −1.18313 | 1.187774 | 1.974228 | −1.83552 | −1.42003 | −1.34451 |
| _ILMN_1245646 | −1.02956 | 1.218253 | 1.413033 | −1.59167 | −1.26363 | −1.02272 |
| Tmem129_ILMN_2429215 | −1.09374 | −1.11964 | −2.07634 | 1.200101 | 1.167903 | 1.342939 |
| Tusc4_ILMN_2454195 | −1.08831 | −1.10728 | −1.85511 | 1.369108 | 1.179966 | 1.089908 |
| Map3k12_ILMN_2725370 | −1.12888 | −1.35818 | −2.18302 | 1.726495 | 1.263127 | −1.07506 |
| Ndrg2_ILMN_2771991 | −1.07046 | −1.1445 | −2.06977 | 1.291192 | 1.095454 | 1.322285 |
| Rrp15_ILMN_2629856 | 1.004134 | −1.02887 | 1.835562 | −1.43385 | −1.48925 | −1.22032 |
| Mum1_ILMN_1215647 | −1.06142 | −1.13778 | −1.74536 | 1.418322 | 1.083703 | 1.103988 |
| AK052106_ILMN_1255302 | −1.05526 | −1.20509 | −1.60772 | 1.39953 | 1.257665 | −1.06004 |
| Tlcd1_ILMN_2781458 | −1.02375 | −1.3448 | −2.10692 | 1.475982 | 1.34724 | 1.0186 |
| Trps1_ILMN_1226073 | −1.06444 | −1.5275 | −1.78979 | 1.757446 | 1.159398 | −1.07492 |
| Ly6a_ILMN_1255416 | −3.17206 | −1.0932 | −1.29711 | 1.461767 | 1.331041 | 1.206246 |
| Accs_ILMN_2776485 | −1.22008 | −1.009 | −1.65779 | 1.41732 | 1.13865 | 1.030122 |
| Appl2_ILMN_1219978 | −1.24559 | −1.27531 | −2.11094 | 1.865122 | 1.161517 | −1.09567 |
| Cox4i2_ILMN_2612178 | 1.089127 | 1.158668 | 1.326957 | 1.008061 | −1.18331 | −1.74794 |
| Clk1_ILMN_1254814 | −1.07911 | −1.22534 | −2.01559 | 1.568467 | 1.193925 | −1.00131 |
| Clspn_ILMN_2623056 | −1.05233 | −1.08879 | −1.82761 | 1.147235 | 1.150539 | 1.28634 |
| Ncrna00166_ILMN_1222196 | −1.03988 | −1.16428 | −1.98635 | 1.39944 | 1.094827 | 1.181747 |
| C4b_ILMN_3049559 | −1.10606 | −1.15706 | −2.05407 | 1.351722 | 1.414119 | −1.0215 |
| Nfatc4_ILMN_2647331 | −1.37338 | −1.25155 | −1.20467 | 1.5856 | 1.183728 | −1.14491 |
| AK036974_ILMN_1222598 | −1.0959 | −1.17652 | −1.59093 | 1.5362 | 1.079376 | −1.00664 |
| Slc5a3_ILMN_1233078 | −1.17885 | −1.23164 | −1.89385 | 1.499145 | 1.458686 | −1.17105 |
| Abcd4_ILMN_1245547 | −1.16716 | −1.03031 | −1.70307 | 1.428297 | 1.110164 | 1.047001 |
| Spnb2_ILMN_1214394 | −1.0152 | −1.12231 | −1.88513 | 1.152147 | 1.231002 | 1.210335 |
| Prelp_ILMN_2739760 | −1.11189 | −1.53571 | −1.7425 | 1.801632 | 1.224774 | −1.17762 |
| 5830411K21Rik_ILMN_1217032 | −1.39544 | −1.00477 | −1.90468 | 1.069172 | 1.653072 | 1.04086 |
| Bmp4_ILMN_1215252 | −1.23614 | −1.17177 | −1.52917 | 1.595397 | 1.230579 | −1.16592 |
| Hoxd8_ILMN_2693052 | −1.07822 | −1.07856 | −1.81581 | 1.155206 | 1.051639 | 1.387814 |
| 1810013L24Rik_ILMN_2616630 | −1.18506 | 1.006997 | −2.02702 | 1.342216 | 1.132491 | 1.18112 |
| 5430432N15Rik_ILMN_2622089 | −1.48775 | −1.75547 | 1.115626 | 1.119898 | 1.119461 | 1.403214 |
| Stxbp3a_ILMN_1245393 | −1.41728 | −1.33413 | −1.13258 | 1.577382 | 1.23735 | −1.18035 |
| Ehd1_ILMN_2628757 | −1.17445 | 1.186862 | 1.650685 | −1.45395 | −1.2074 | −1.25788 |
| Raf1_ILMN_1237730 | −1.07621 | −1.19935 | −1.61396 | 1.5239 | 1.171328 | −1.08435 |
| Tbc1d2b_ILMN_2819859 | −1.07046 | −1.08129 | −2.17266 | 1.290826 | 1.221243 | 1.168665 |
| Ppox_ILMN_2826816 | −1.06553 | −1.21576 | −1.96091 | 1.443881 | 1.371369 | −1.09439 |
| Eraf_ILMN_2619200 | −1.14473 | 1.597198 | 1.416693 | −2.1079 | 1.035463 | −1.65928 |
| Hbb-b1_ILMN_1244316 | 1.271854 | 1.441461 | 1.216162 | −2.80858 | 1.193409 | −1.91916 |
| Chst5_ILMN_2665754 | 1.01614 | −1.33756 | −1.72423 | 1.103901 | −1.00597 | 1.558293 |

Dataset 2: List of transcription factors predicted by Ingenuity Pathway Analysis to be activated or inhibited. Positive z score suggests activation and negative z score.

| Transcription | Regulation z- | | | |
|---|---|---|---|---|
| Regulation | score | p-value of overlap | Target molecules in dataset | Molecular Type |
| PPARA | 2.196 | 1.89E−01 | ASNS, CHKA, Clec2d (includes | ligand-dependent nuclear receptor |
| | | | others), CPT2, FABP3, HIST1H1C, IGFBP5, | |
| | | | KIF2C, LGALS4, RETSAT, SRM, TOP2A | |
| STAT5B | 2.038 | 1.42E−01 | MYL2, TNNC1, TNNT1, TPM3, TROVE2 | transcription regulator |
| PPARD | 2.036 | 1.29E−01 | ACTG2, CPT2, FABP3, FN1, LGALS4, | ligand-dependent nuclear receptor |
| | | | TNFRSF12A | |
| ESR1 | 2.021 | 3.42E−01 | BMP4, Clec2d (includes | ligand-dependent nuclear receptor |
| | | | others), DDX21, HOXC6, IER3, IGFBP5, | |
| | | | SGK1, TGFB3 | |
| FOX03 | −2.188 | 2.97E−01 | IER3, PPP1R15A, SGK1, SLC1A4 | transcription regulator |
| MEF2C | −2.359 | 2.06E−02 | Bglap (includes | transcription regulator |
| | | | others), IBSP, MYL2, TNNC1 | |
| FOS | −2.58 | 3.37E−01 | CASZ1, CDON, FN1, HBA1/HBA2, HBB, | transcription regulator |
| | | | IBSP, IGFBP5, KIF1B, LGALS4, NFATC1, | |
| | | | S100A8, S100A9, SIRPA | |
| GATA4 | −2.64 | 1.61E−02 | ACTC1, ACTG2, MYLA, MYLPF, TNNC1 | transcription regulator |
| MY0CD | −2.976 | 1.88E−04 | ACTG2, GJA5, LPP, MYL2, MYL4, MYLPF | transcription regulator |
| SRF | −3.454 | 1.05E−03 | ACTC1, ACTG2, GADD45G, HIF3A, LBH, | transcription regulator |
| | | | MYL2, MYL3, MYL4, Myl9, MYLPF, RAF1, | |
| | | | TNNC1, TNNT1, ZMYM2 | |
| GLI1 | 1.845 | 1.78E−01 | LMNA, NDRG2, PDGFRA, S100A9, | transcription regulator |
| | | | TMEM100, WIF1 | |
| E2F1 | 1.844 | 3.83E−01 | BMP4, DDX11/DDX12P, MCM10 (includes | transcription regulator |
| | | | EG: 307126), NRP1 (includes | |
| | | | EG: 18186), POLD1, PRDM2, RAD54L, | |
| | | | TK1, TOP2A | |
| SATB1 | 0.865 | 3.41E−02 | ABTB1, HBB, HSPA8, SGK1, TSC22D3, | transcription regulator |
| | | | YPEL5 | |
| CEBPD | 0.795 | 3.70E−02 | ASNS, IGFBP5, MBP, MIA, PDGFRA | transcription regulator |
| TFAP2C | 0.427 | 3.11E−03 | HIST1H1C, MBP, NRP1 (includes | transcription regulator |
| | | | EG: 18186, TK1, ZMYND11 | |
| SMARCB1 | 0.271 | 3.27E−02 | C4B (includes others), CDC7 (includes | transcription regulator |
| | | | EG: 12545), KIF23, MCM10 (includes | |
| | | | EG: 307126), PLXNB2, PPP1R3C.RAB5B, | |
| | | | RAD54B | |
| RUNX2 | −0.09 | 7.90E−03 | ACTG2, Bglap (includes others), C4B | transcription regulator |
| | | | (includes others), COL24A1, FN1, IBSP | |
| SMAD7 | −0.265 | 4.16E−02 | ACTG2, CITED2, FN1, MYLPF, TGFB3, | transcription regulator |
| | | | TPM3 | |
| PGR (includes | −0.275 | 5.84E−03 | CPT2, DDX21, IER3, IGFBP5, NEDD9, | ligand-dependent nuclear receptor |
| EG: 18667) | | | SRSF7, TGFB3, TK1, TSC22D3 | |
| TP53 (includes | −0.709 | 1.68E−03 | ASNS, BMP1, CCDC80, CDC7 (includes | transcription regulator |
| EG: 22059) | | | EG: 12545), CSNK1D, DBP, FABP3, FN1, | |
| | | | GADD45G, GSTM1, HDC, HJURP, HK2, HSPA8, | |
| | | | IER3, EGFBP5, IKBIP, IQCB1, KIF23, | |
| | | | LPP, Ly6a (includes | |
| | | | others), MYL4, Myl9, NDRG2, NRP1 | |
| | | | (includes EG: 18186), | |
| | | | PDGFRA, PEG3, PLNB2, POLD1, | |
| | | | PPP1R15A, PQLC3, RAD54B, RAF1, | |
| | | | RNASE4, SESN1, SGK1, TOP2A, TSC22D3 | |
| CEBPB (includes | −1.133 | 2.42E−02 | ACTG2, ARPP19, ASNS, CIRBP, Gnas | transcription regulator |
| EG: 1051) | | | (mouse), HBB, HDC, IER3, MBP, MIA, NRP1 | |
| | | | (includes EG: 18186), PDGFRA, PPP1R15A, SGK1 | |
| MITF | −1.373 | 3.65E−02 | CHKA, CMA1, EDNR8, GPNM8, MBP, MYL4, | transcription regulator |
| | | | Tpsab1 | |
| KDM5B | −1.459 | 1.71E−02 | A | transcription regulator |
| GATA1 | −1.697 | 1.36E−02 | AHSP, ALAS2, CP1BB, HBA1/HBA2, HBB, | transcription regulator |
| | | | MBP | |
| HIF1A | −1.714 | 3.60E−03 | ACTG2, ASNS, CHKA, CITED2, FN1, HIF3A, | transcription regulator |
| | | | HIST1H1C, HK2, 1GFBPS, MEF, PFKL, | |
| | | | SLC29A1, TGFBB, TMEM128 | |
| SMARCA4 | −1.791 | 9.94E−03 | ACTG2, ASNS, Bglap (includes | transcription regulator |
| | | | others), BMP4CLK1, FN1, HBB, IGFBPS, | |
| | | | LMNA, MYH3, MYL4, MYLPF, NRP1 | |
| | | | (includes EG: 18186) | |
| MYOD1 | −1.839 | 6.25E−03 | ACTC1, ACTG2, GADD45G, IGFBPS, MYH3, | transcription regulator |
| | | | MYL4, MYLPF, SPTBN1, TNNC1, TNNT1 | |
| | | | ASNS, Bglap (includes | |
| | | | others), IBSP, IGFBPS, PPP1R15A, | |
| | | | SLC7A5, TNFR | |
| ATF4 | −1.848 | 7.63E−03 | SF12A | transcription regulator |

Sequence Listing Overview:

| | |
|---|---|
| SEQ ID NO 1 | HDAd-huPRG4 |
| SEQ ID NO 2 | HDAd-muPRG4 |
| SEQ ID NO 3 | huPRG4 |
| SEQ ID NO 4 | muPRG4 |
| SEQ ID NO 5 | huPRG4 protein |
| SEQ ID NO 6 | muPRG4 protein |
| SEQ ID NO 7 | HDAd-huIL1Ra |
| SEQ ID NO 8 | HDAd-mull1Ra |
| SEQ ID NO 9 | HDAd-eqll1Ra |
| SEQ ID NO 10 | hull1Ra |
| SEQ ID NO 11 | mull1Ra |
| SEQ ID NO 12 | eqll1Ra |
| SEQ ID NO 13 | hull1Ra protein |
| SEQ ID NO 14 | mull1Ra protein |
| SEQ ID NO 15 | eqll1Ra protein |

hu = human;
mu = murine;
eq = equine

What is claimed is:

1. A gene therapy delivery and expression system, comprising at least one helper-dependent adenoviral vector containing a nucleic acid sequence encoding proteoglycan 4 (PRG4), or a biologically active fragment thereof, which has chondoprotective activity, left and right adenoviral inverted terminal repeats (L ITR and R ITR), adenoviral packaging signal sequences and non-viral, non-coding stuffer nucleic acid sequences, wherein PRG4 expression in the at least one helper-dependent adenoviral vector is controlled by a ubiquitous, constitutive promoter, wherein
i. the helper-dependent adenoviral vector additionally comprises a nucleic acid sequence encoding one or more inhibitors of inflammatory and cartilage destructive mediators, or
ii. the delivery and expression system comprises a second helper-dependent adenoviral vector comprising a nucleic acid sequence encoding one or more inhibitors of inflammatory and cartilage destructive mediators.

2. The gene therapy delivery and expression system according to claim 1, wherein the one or more inhibitors of inflammatory and cartilage destructive mediators are selected from a cytokine, leukemia inhibitory factor (LIF), oncostatin M, a matrix metalloprotease, an aggrecanase, a toll-like receptor and nuclear factor ‘kappa-light-chain-enhancer’ of activated B-cells (NF-κB).

3. The gene therapy delivery and expression system according to claim 2, wherein
(a) the cytokine is selected from Il-1, TNFa, Il-6, Il-7, Il-8, Il-11, Il-15, Il-17, Il-18, and Il-21;
(b) the matrix metalloprotease is selected from MMP-1, MMP-3, MMP-9, and MMP-13;
(c) the aggrecanase is selected from ADAMTS-1, ADAMTS-4, and ADAMTS-5; and/or
(d) toll-like receptor is selected from TLR2 and TLR4.

4. The gene therapy delivery and expression system according to claim 1, wherein the one or more inhibitors of inflammatory or cartilage destructive mediator is interleukin-1 receptor antagonist (Il-1Ra).

5. The gene therapy delivery and expression system according to claim 1, wherein the ubiquitous constitutive promoter is selected from the group consisting of elongation factor 1 alpha (EF1 alpha) promoter, cytomegalovirus (CMV) promoter, beta-actin promoter, simian virus 40 (SV40) early promoter, ubiquitin c promoter, glyceraldehyde 3-phosphate dehydrogenase (GAPDH) promoter, phosphoglycerate kinase (PGK) promoter.

6. The gene therapy delivery and expression system according to claim 1, wherein the helper-dependent adenoviral vector containing a nucleic acid sequence encoding proteoglycan 4 (PRG4) comprises a nucleic acid sequence which has at least 80% or 90% sequence homology with a nucleic acid sequence set forth in SEQ ID NO 1, or SEQ ID NO 2, or a biologically active fragment thereof, or a homolog from any other species.

7. The gene therapy delivery and expression system according to claim 1, wherein the nucleic acid sequence encoding proteoglycan 4 (PRG4) comprises a nucleic acid sequence which has at least 80% or 90% sequence homology with a nucleic acid sequence set forth in SEQ ID NO 3, or SEQ ID NO 4, or a biologically active fragment thereof, or a homolog from any other species.

8. The gene therapy delivery and expression system according to claim 1, wherein the amino acid sequence of proteoglycan 4 (PRG4) comprises an amino acid sequence which has at least 80% or 90% sequence homology with an amino acid sequence set forth in SEQ ID NO 5, or SEQ ID NO 6, or a biologically active fragment thereof, or a homolog from any other species.

9. The gene therapy delivery and expression system according to claim 4, wherein expression of interleukin-1 receptor antagonist (Il-1Ra) is controlled by an inflammation-inducible promoter selected from the group consisting of NF-κB promoter, interleukin 6 (Il-6) promoter, interleukin-1 (Il-1) promoter, tumor necrosis factor (TNF) promoter, cyclooxygenase 2 (COX-2) promoter, complement factor 3 (C3) promoter, serum amyloid A3 (SAA3) promoter, macrophage inflammatory protein-1α (MIP-1α) promoter, or hybrid constructs of the above.

10. The gene therapy delivery and expression system according to claim 4, wherein the helper-dependent adenoviral vector comprising the nucleic acid sequence encoding interleukin-1 receptor antagonist (Il-1Ra), comprises a nucleic acid sequence which has at least 80% or 90% sequence homology with a nucleic acid sequence set forth in SEQ ID NO 7, or SEQ ID NO 8, SEQ ID NO 9, or a biologically active fragment thereof, or a homolog from any other species.

11. The gene therapy delivery and expression system according to claim 4, wherein the nucleic acid sequence encoding interleukin-1 receptor antagonist (Il-1Ra) comprises a nucleic acid sequence which has at least 80% or 90% sequence homology with a nucleic acid sequence set forth in SEQ ID NO 10, or SEQ ID NO 11, or SEQ ID NO 12, or a biologically active fragment thereof, or a homolog from any other species.

12. The gene therapy delivery and expression system according to claim 4, wherein the amino acid sequence of interleukin-1 receptor antagonist (Il-1Ra) comprises an amino acid sequence set forth in SEQ ID NO 13, SEQ ID NO 14, SEQ ID NO 15, or a biologically active fragment thereof, which has Il1-Ra activity of inhibiting inflammatory and cartilage destructive mediators.

13. A pharmaceutical composition, comprising a therapeutically effective amount of at least one helper-dependent adenoviral vector containing a nucleic acid sequence encoding proteoglycan 4 (PRG4), or a biologically active fragment thereof, which has chondoprotective activity, left and right adenoviral inverted terminal repeats (L ITR and R ITR), adenoviral packaging signal sequences and non-viral, non-coding stuffer nucleic acid sequences, wherein PRG4 expression in the at least one helper-dependent adenoviral vector is controlled by a ubiquitous, constitutive promoter, wherein
i. the helper-dependent adenoviral vector additionally comprises a nucleic acid sequence encoding one or more inhibitors of inflammatory and cartilage destructive mediators, or
ii. the delivery and expression system comprises a second helper-dependent adenoviral vector comprising a nucleic acid sequence encoding one or more inhibitors of inflammatory and cartilage destructive mediators.

14. The pharmaceutical composition according to claim 13, wherein the one or more inhibitors of inflammatory and cartilage destructive mediators are selected from a cytokine, leukemia inhibitory factor (LIF), oncostatin M, a matrix metalloprotease, an aggrecanase, a toll-like receptor and nuclear factor ‘kappa-light-chain-enhancer’ of activated B-cells (NF-κB).

15. The pharmaceutical composition according to claim 14, wherein
(a) the cytokine is selected from Il-1, TNFa, Il-6, Il-7, Il-8, Il-11, Il-15, Il-17, Il-18, and Il-21;
(b) the matrix metalloprotease is selected from MMP-1, MMP-3, MMP-9, and MMP-13;
(c) the aggrecanase is selected from ADAMTS-1, ADAMTS-4, and ADAMTS-5; and/or
(d) toll-like receptor is selected from TLR2 and TLR4.

16. The pharmaceutical composition according to claim 13, wherein the one or more inhibitors of inflammatory or cartilage destructive mediator is interleukin-1 receptor antagonist (Il-1Ra).

17. The pharmaceutical composition according to claim 13, wherein the ubiquitous constitutive promoter is selected from the group consisting of elongation factor 1 alpha (EF1 alpha) promoter, cytomegalovirus (CMV) promoter, beta-actin promoter, simian virus 40 (SV40) early promoter, ubiquitin c promoter, glyceraldehyde 3-phosphate dehydrogenase (GAPDH) promoter, phosphoglycerate kinase (PGK) promoter.

18. The pharmaceutical composition according to claim 13, wherein the helper-dependent adenoviral vector containing a nucleic acid sequence encoding proteoglycan 4 (PRG4) comprises a nucleic acid which has at least 80% or 90% sequence homology with a nucleic acid sequence set forth in SEQ ID NO 1, or SEQ ID NO 2, or a biologically active fragment thereof, or a homolog from any other species.

19. The pharmaceutical composition according to claim 13, wherein the nucleic acid sequence encoding proteoglycan 4 (PRG4) comprises a nucleic acid sequence which has at least 80% or 90% sequence homology with a nucleic acid sequence set forth in SEQ ID NO 3, or SEQ ID NO 4, or a biologically active fragment thereof, or a homolog from any other species.

20. The pharmaceutical composition according to claim 13, wherein the amino acid sequence of proteoglycan 4 (PRG4) comprises an amino acid sequence which has at least 80% or 90% sequence homology with an amino acid sequence set forth in SEQ ID NO 5, or SEQ ID NO 6, or a biologically active fragment thereof, or a homolog from any other species.

21. The pharmaceutical composition according to claim 16, wherein expression of interleukin-1 receptor antagonist (Il-1Ra) is controlled by an inflammation-inducible promoter selected from the group consisting of NF-κB promoter, interleukin 6 (Il-6) promoter, interleukin-1 (Il-1) promoter, tumor necrosis factor (TNF) promoter, cyclooxygenase 2 (COX-2) promoter, complement factor 3 (C3) promoter, serum amyloid A3 (SAA3) promoter, macrophage inflammatory protein-1α (MIP-1α) promoter, or hybrid constructs of the above.

22. The pharmaceutical composition according to claim 16, wherein the helper-dependent adenoviral vector comprising the nucleic acid sequence encoding interleukin-1 receptor antagonist (Il-1Ra), comprises a nucleic acid sequence which has at least 80% or 90% sequence homology with a nucleic acid sequence set forth in SEQ ID NO 7, or SEQ ID NO 8, SEQ ID NO 9, or a biologically active fragment thereof, or a homolog from any other species.

23. The pharmaceutical composition according to claim 16, wherein the nucleic acid sequence encoding interleukin-1 receptor antagonist (Il-1Ra) comprises a nucleic acid sequence set forth in SEQ ID NO 10, or SEQ ID NO 11, or SEQ ID NO 12, or a biologically active fragment thereof, or wherein the nucleic acid sequence encoding for interleukin-1 receptor antagonist (Il-1Ra) comprises a nucleic acid sequence which has at least 80% or 90% sequence homology with a nucleic acid sequence set forth in SEQ ID NO 10, or SEQ ID NO 11, or SEQ ID NO 12, or a biologically active fragment thereof, or a homolog from any other species.

24. The pharmaceutical composition according to claim 16, wherein the amino acid sequence of interleukin-1 receptor antagonist (Il-1Ra) comprises an amino acid sequence set forth in SEQ ID NO 13, SEQ ID NO 14, SEQ ID NO 15 or a biologically active fragment thereof, or a homolog from any other species.

25. A method for treating or preventing camptodactyly-arthropathy-coxa vara-pericarditis (CACP) syndrome in a subject, comprising administering to the subject a therapeutically effective amount of the gene therapy delivery and expression system according to claim 1.

26. A method for treating or preventing a musculoskeletal disorder or joint disorder in a subject, comprising administering to the subject a therapeutically effective amount of the gene therapy delivery and expression system according to claim 1 wherein the musculoskeletal disorder or joint disorder is selected from the group consisting of arthropathies, arthritis, an arthritis-related disorders, osteoarthritis, rheumatoid arthritis, gout, pseudo-gout, septic arthritis, psoriatic arthritis, ankylosing spondylitis, juvenile idiopathic arthritis, Still's disease, Reiter's syndrome, a tendinopathy including tendonitis, tendinosis, tenosynovitis; synovial disorders including synovitis; Bursa disorders including bursitis; equine musculoskeletal disorders including bone spavin, navicular syndrome, and osselet.
